(19) United States
(12) Patent Application Publication
(10) Pub. No.: US 2013/0071839 A1
(43) Pub. Date: Mar. 21, 2013

(54) SYSTEMS AND METHODS FOR DETECTING BIOMARKERS OF INTEREST

(71) Applicants: Georg Seelig, Seattle, WA (US); Barry Lutz, Seattle, WA (US)

(72) Inventors: Georg Seelig, Seattle, WA (US); Barry Lutz, Seattle, WA (US)

(21) Appl. No.: 13/603,298

(22) Filed: Sep. 4, 2012

Related U.S. Application Data
(60) Provisional application No. 61/530,739, filed on Sep. 2, 2011.

(51) Int. Cl. C12Q 1/68

(52) U.S. Cl. CPC C12Q 1/6876; Y10S 977/81; Y10S 977/902
USPC 435/6.11; 977/810; 977/902

(57) ABSTRACT

In some embodiments, a strand displacement system is provided. Such a system may include a first nucleic acid catalyst molecule; a nucleic acid gate molecule, wherein the first nucleic acid catalyst molecule binds the nucleic acid gate molecule forming a nucleic acid gate-catalyst complex and releases an output molecule; and a nucleic acid sink molecule. The nucleic acid sink molecule sequesters a putative second nucleic acid catalyst, wherein the second nucleic acid catalyst differs from the first nucleic acid catalyst molecule by at least one nucleotide. In some aspects, the first nucleic acid catalyst may include a biomarker of interest or a nucleic acid aptamer which binds an amino acid-based biomarker of interest.

### PRIORITY CLAIM

This application claims priority to U.S. Provisional Patent Application Ser. No. 61/530,739, filed Sep. 2, 2011, the subject matter of which is hereby incorporated by reference as if fully set forth herein.

### STATEMENT OF GOVERNMENT INTEREST

The present invention was made with government support under Grant No. CBET-1041548, awarded by the National Science Foundation (NSF). The Government has certain rights in the invention.

### BACKGROUND

Pathogen-derived proteins and nucleic acids have important roles as diagnostic markers for infectious diseases. For example, the detection of just a single molecular marker, such as a DNA sequence associated with the M. tuberculosis complex, can sometimes be indicative of a disease state. However, a reliable diagnosis and treatment decision often requires interpreting a combination of markers via complex algorithms. For example, in the case of DNA testing for tuberculosis (TB), the treatment decision requires interpretation of markers associated with the M. tuberculosis complex, TB antibiotic resistance and even HIV-coinfection (McNerney et al., 2011).

DNA-based circuits and reaction networks may be designed that can be used for the analysis of complex molecular mixtures. Synthetic molecular circuits that are capable of information processing and computation have been built using a range of approaches. Examples include synthetic gene regulatory and signaling networks (Isaacs et al., 2006; Yeh et al., 2007; Win et al., 2008), computational networks using in vitro transcription (Kim et al., 2006; Simpson et al., 2009), digital logic circuits based on small molecules (de Silva et al., 2007) or peptides (Ashkenasy et al., 2004), and the nonlinear chemical reaction networks underlying the Belousov Zhabotinskii reaction and related phenomena (Epstein et al., 1998). In these circuits, information is stored in the concentrations, spatial localizations, and/or chemical properties of molecules; chemical reactions between molecules implement molecular information processing. Most of these systems lack the flexibility and modularity that would make them useful for biosensing applications. For proteins and small molecules in particular, the de novo design of individual functional molecular sensors and logic gates is difficult and integration of multiple elements into circuits is even more challenging.

Because of the predictability of Watson-Crick base pairing, nucleic acid-based systems avoid some of these constraints and can be used to implement modular and scalable molecular computation. Initial demonstrations of nucleic acid logic circuits took advantage of enzyme or deoxyribozyme catalysis (Lu et al., 2006; Willner et al., 2008). Also, a DNA and enzyme-based molecular automaton was developed that could perform a computation where the outcome (the release of an antisense drug mimic) was dependent on the absence or presence of specific inputs (ssDNA with sequence analogous to diagnostically relevant mRNA) (Benenson et. al., 2001; Benenson et al., 2004). Stojanovic and collaborators developed deoxyribozyme based logic gates (Stojanovic et al, 2002) and used these gates to form a variety of logic circuits (Stojanovic et al., 2003; Lederman et al, 2006; Yashin et al., 2007). Penchovsky and Breaker (Penchovsky et al., 2005) developed allosteric ribozymes that could implement cascaded logic using DNA inputs and RNA outputs. More recent work (Takahashi et al, 2006; Frezza et al., 2007; Cardelli et al., 2008; Qian et al., 2011; Seelig et al., 2006; Soloveichik et al., 2010), has relied on hybridization and strand displacement as a mechanism for implementing molecular logic.

Although the field of DNA-based circuits and reaction networks has several promising approaches, such approaches are limited by their ability to discriminate between closely related molecules, especially when the sequence of the related molecules differs by a small number nucleotides. Thus, it would be desirable to design a system capable of robustly distinguishing molecules having related or similar sequences.

### SUMMARY

In one embodiment, a strand displacement system is provided. Such a system may include a first nucleic acid catalyst molecule; a nucleic acid gate molecule, wherein the first nucleic acid catalyst molecule binds the nucleic acid gate molecule forming a nucleic acid gate-catalyst complex and releases an output molecule; and a nucleic acid sink molecule. The nucleic acid sink molecule sequesters a putative second nucleic acid catalyst, wherein the second nucleic acid catalyst differs from the first nucleic acid catalyst molecule by at least one nucleotide. In some embodiments, the first nucleic acid catalyst includes a biomarker of interest, such as a DNA or RNA molecule. In other embodiments, the first nucleic acid catalyst is a nucleic acid aptamer which binds an amino acid-based biomarker of interest. The system may also include a nucleic acid fuel molecule, wherein the nucleic acid fuel molecule binds the nucleic acid gate/catalyst complex and releases the first nucleic acid catalyst molecule.

In another embodiment, the strand displacement system may be part of a paper-based diagnostic tool. The paper-based diagnostic tool may include a paper device that comprises a set of reaction components attached to at least one reaction zone, wherein the set of reaction components includes a nucleic acid gate molecule and a nucleic acid sink molecule; and a set of output capture probes attached to at least one detection zone, wherein the output capture probes are complementary to an output molecule released by the nucleic acid gate molecule.

In other embodiments, methods for detecting a biomarker of interest in a biological sample are provided. Such methods may include a step of exposing the biological sample which contains or is suspected of containing a first nucleic acid catalyst molecule to a reaction zone of a paper-based diagnostic tool, wherein the first nucleic acid catalyst molecule comprises the biomarker of interest and the reaction zone comprises a set of reaction components which includes a nucleic acid gate molecule and a nucleic acid sink molecule, and. The methods may further include a step of detecting the biomarker of interest by visualizing a change in signal in a detection zone of the paper-based diagnostic tool, wherein the change in signal is produced when an output molecule binds a nucleic acid capture molecule that is attached to the detection zone

### BRIEF DESCRIPTION OF THE DRAWINGS

FIG. 1 illustrates examples of double-stranded toehold exchange probes. (A) shows that hybridization is the binding process of complementary single-stranded nucleic acids. Formation of base pairs provides energy gain during hybridization. When there is a mismatch, the system will gain less energy, but the reaction still goes forward. (B) shows an example of strand displacement, which is the process of using one strand to displace another one. The single-stranded input binds to a double-stranded complex using partially single-stranded regions which are called toeholds (green). Because the black regions are identical for both top strands, a three way branch migration will start. The top strand, which originally binds the bottom strand, will fall off. (C) by adding a toehold on the other end (blue), the strand that fell of in (B) may re-bind the bottom strand, making the reaction reversible. This binding exchange is called toehold exchange. During this process, there is almost no enthalpy change and very few entropy change independent of environment. This method has been used to characterize nucleic acid dynamics, build catalysts, and make specific detection probes. (D) shows a four way strand displacement, which happens between four single-stranded nucleic acid and two sets of binding. The junction between four strands is called a holliday junction, and it represents an intermediate stage in genetic recombination.

FIG. 2 illustrates how strand displacement can be used to detect single-stranded nucleic acids, according to some embodiments. (A) A strand displacement reaction where an input strand (1:2:3) displaces an output strand (2:3:4) from a gate/sensor complex. DNA is represented as directional lines, with the arrow denoting the 3′ end. Domains are labeled by numbers, with _ denoting Watson-Crick complementarity. Multiple elementary steps are indicated: (1) binding of toeholds 1 and 1*; (2) a random walk branch migration process where domain 2 of strand 2: 3: 4 is partially displaced by domain 2 of strand 1: 2: 3; (3) the separation of domains 3 and 3* and release of an output strand. (B) The output strand of one strand displacement reaction can serve as an input to a downstream reaction. In this case, a reporter complex is used to detect the output strand released in the upstream reaction. The two strands in the reporter complex are chemically labeled with a fluorophore (magenta star) and a quencher (dark blue dot), respectively. Separation of the fluorophore and quencher lead to an increase in fluorescence. (C) Example kinetics traces for the two-step reaction sketched in (A) and (B). Gate complex is at 10 nM, readout at 13 nM, input concentration is varied from 0 nM to 10 nM. Reactions are run at 25° C. in TAE buffer with 12.5 mM Mg++. Domain 1 is 5 nucleotides (nt), domains 2 and 4 are 15 nt and domain 3 is 6 nt. The data can be fit well with a simple model assuming two sequential bimolecular reactions. (D) Strand displacement kinetics strongly depends on the free energy released when domains 1 and 1* hybridize. Reaction rates increase over several orders of magnitude as the toehold length is increased from 0-6 nt.

FIG. 3 illustrates a catalytic system that includes a fuel molecule. The reaction starts with the toehold binding of the Gate and the Input (catalyst), followed by branch migration. The Output strand will be released from the Gate through branch migration, and the hidden toehold is exposed. A single-stranded Fuel molecule may bind on the exposed toehold and release the Input molecule. The Input can then be reused to trigger release of more Output strands to enhance the signal. The Output strands may react with a reporter to produce a signal.

FIG. 4 illustrates that a seesaw gate may be used in a system for catalytic signal amplification, in accordance with some embodiments, which provides the basis for the biosensing systems provided herein. (A) The amplifier includes one double-stranded complex (“gate”) and an auxiliary strand “fuel.” The catalyst strand initiates the reaction by binding the gate through toehold mediated strand displacement. In the Examples provided herein, the role of the catalyst is played by a single-stranded biomarker of interest. (B) The reaction mechanism for the seesaw gate is initiated by the catalyst. The catalyst (1:2) binds the double stranded gate, resulting in release of the output (2:3:4). Next, the fuel strand (2:3) binds and releases the catalyst. See Qian et al., 2011 for more details regarding the seesaw gate system. (C) In this catalytic signal amplification experiment, the gate and fuel are at 10 nM and the catalyst concentration is varied. A readout gate is used to detect release of the output strand. The concentration of the readout is 13 nM.

FIG. 5 shows a logic circuit using a combination of microRNA and related precursor inputs and corresponding gates. (A) Signal propagation through an in vitro chemical circuit combining AND, OR, sequence translation, input amplification, and signal restoration. The five-layer circuit includes a total of 11 gates and accepts six inputs. Corresponding DNA structures are shown with the circuit diagram (inset). (B) Fluorescence traces of circuit operation without and with the signal restoration module (threshold plus amplifier). The traces for input conditions corresponding to a logical TRUE output (ON) are clearly distinguishable from the logical FALSE output (OFF). Cases tested include when all inputs are present, all cases in which exactly one input is missing, and combinations of inputs that turn off an OR clause.

FIG. 6 is a schematic diagram showing a variety of diagnostic tests available, including lateral flow tests, instrumented PCR, line probe assays, and DNA/RNA microarrays. The diagnostics tests are arranged in accordance with their strengths and suitable use. Tests that are show farthest to the right are most suitable for a large numbers of markers that can improve diagnosis and provide comparative results, but require complex interpretation algorithms. Those shown toward the left are most suitable for use with single biomarkers and provide for easy to use, rapid results.

FIG. 7 shows a series of multi-step processes in paper networks, according to some embodiments. (A) Sugar solutions dried on each leg create fluidic time delays that can be controlled by the sugar concentration. Each fluid source has a limited volume so it shuts off at a programmed time. The result is delivery of multiple fluids to a detection zone in a timed sequence. (B) A folding card design is used to contact fluid source pads to the network for a single step initiation of the fluid sequence. (C) Fluid from each leg is delivered in a timed sequence on a time scale appropriate for rapid point of care assays (30 minutes). Reagents can be stored in dry form on the pads, such that the user only adds a sample and buffer or water. Alternative designs have been used to perform automated assay sequences for wash steps and chemical signal amplification.

FIG. 8 shows a mechanism of detecting mismatch discrimination according to some embodiments. (A) A combination of a seesaw amplifier and a “sink” complex can be used to distinguish catalyst strand that differs by only a single base. (B) A seesaw amplifier without a sink can be used for kinetic discrimination of the inputs. However, end-point discrimination is not possible. Amplifier components are 10 nM, readouts are 13 nM and all catalysts are 5 nM. Experiments are performed in TAE with 12.5 mM Mg++. (C) The input can be inhibited by addition of a sink. When the input binds the sink and completes branch migration, the input will not be released again. (D) Gates and sinks have different binding affinities for binding correct and mismatched targets. The correct input (catalyst) binds to the amplifier gate faster than it binds to the inhibitory sink. Although the input may be degraded slowly by the sink, a fast catalytic reaction of the input and gate ensures completion of amplification. The mutated input (catalyst with mismatched mutation) will be inhibited rapidly and thus almost no signal will be triggered. (E) Combining the seesaw amplifier with a sink enables endpoint discrimination. Reaction conditions are similar to those in (b). The sink concentration is 10 nM. (F) Kinetics with and without the mismatch can be predicted using binding energy. Reactions involving mis-binding are significantly slower. For idea rapid endpoint discrimination, the correct catalyst toehold binding should have energy around 10 kcal/mol.

FIG. 9 shows a system for detection of DNA analogs of the let-7 family according to some embodiments. (A) The let-7 family is a set of miRNAs that have very similar sequences (SEQ ID NOs:1-9). (B) Experimental results from the let-7 detection system indicate that the catalytic system for let-A can be triggered by several other let-7 mismatched inputs. (C) The same catalytic system as shown in (B) is prepared using various sinks to sequester the mismatched let-7 family members. When sinks are added to the reaction, clear endpoint discrimination is seen for correct and mismatched inputs. With the sink, mismatched catalysts are degraded, producing little signal which is similar to that seen with a system leak. (D) Two catalytic systems, let-A and let-C, were run in parallel using different output channels, the results of which are shown in (E).

FIG. 10 illustrates double-stranded toehold exchange probes. (A) shows a double toehold exchange probe P (or PtPb), which includes a fork shaped toehold. Such toeholds help initiate four-way branch migration. In this case, 5 new base pairs (orange and purple) are formed through the course of the reaction, and 4 base pairs (blue and green single-stranded regions) and a fluorophore-quencher interaction are broken. Toeholds before and after the reaction have similar lengths so that the standard free energy of the forward reaction is about 0 kcal/mol. Thus, when the correct target reacts with the probe, roughly equal amounts of reactant and products are seen at equilibrium. (B) shows a double-stranded toehold exchange reaction with a spurious target S (or StSb). T his forward four-way branch migration at the mutated position is very slow and not favorable due to the energy penalty caused by mismatch binding. The intermediate step shown possesses a much higher backward reaction rate constant. Consequently, there should be much fewer products at equilibrium.

FIG. 11 shows the results of a fluorescence assay. (A) shows sequences of correct and single-base-changed targets, and a corresponding probe. The probe is functionalized with the ROX fluorophore and the Iowa Black Red Quencher. (B) shows the kinetic traces of the reaction between the probe and the correct target (top) or spurious insertion target (bottom) with various concentrations of target. In all traces, [PtPb]=10 nM. (C) shows a log-log plot of hybridization yield X as a function of the concentration of the target. Dots represent experimental values of X, and lines represent the analytic relationship between X and [Target], using a least-squares fit for the y-intercept (X at equal stoichiometry). Discrimination factor (Q) is defined as Q=(X_{correct})/(X_{mutated}), and concentration tolerance (R) is defined as R=([TtTb]_{X=0.5})/([StSb]_{X=0.5}), the ratio of concentrations needed to achieve 50% yield at equilibrium.

FIG. 12 shows robustness over mutation position identities, as well as temperature and salinity. (A) shows tested mutation position and identities. (B) shows a kinetics trace for all correct targets (Correct) and mutated targets (All SNPs). The mutated targets give little to no signal. (C) shows the discrimination factor (Q) as measured for the targets shown. The discrimination factor is defined as Q=(X_{correct})/(X_{mutated}), and has a median of 43. (D) shows that mutated target m8G-C was tested under different temperature and salinity.

FIG. 13 shows the detection of a mutation in the RpoB gene from M. tuberculosis (TB). (A) shows the subsequence of TB (SEQ ID NO:9). Two systems of different length and sources were tested. The short 50 nt target was made synthetically, and the 100 nt target was made from a plasmid containing the subsequence of TB using PCR. (B) shows testing of one mutation at position 526 with a s50 nt TB subsequence under various concentrations as shown. (C) shows single strands with a desired toehold end could be generated from unbalanced PCR, wherein one primer has 100 times lower concentration than ordinary PCR. Annealing the two single strands, the double strand with toeholds may be generated. (D) shows the results of testing the purified annealing product.

FIG. 14 shows various methods for diagnosing TB antibiotic resistance according to some embodiments. (A) An example of TB targets and interpretation algorithm (partial set) for identification of resistance (RMP: rifampin, INH: isoniazid). Adapted from the Genotype MTBDR+ line probe assay (Niemz et al., 2011). (B) A logic circuit diagram for a diagnostic circuit for detection of different markers associated with TB antibiotic resistance. The circuit combines an embedded analysis with a simplified readout.

FIG. 15 shows a schematic of a two-stage strip for sequencing DNA circuit reactions and visually detecting output DNA according to some embodiments. Labeled outputs that are prebound with immobilized DNA circuit elements are only released when the DNA circuit is completed by input DNA. These labeled outputs are released by a fluidic timer to be captured by hybridization for visual detection. Fluidic timers are made from dried sugar; time delays are adjustable from seconds to an hour.

FIG. 16 shows the length of paper-based fluidic delays as a function of percentage of dried sugar according to some embodiments. (A) Delays are created by dipping strips into sugar solutions with different concentrations, followed by drying; delays from minutes to over an hour are possible. A relative delay of 10 equates to approximately 300 seconds for the strips used here. (B) Sugar solutions pipetted onto a paper strip create delays used to stage fluids in different reaction zones.

### DETAILED DESCRIPTION

Systems or “logic circuits” for detecting a biomarker of interest and methods for their use are provided herein. According to the embodiments described herein, such systems may include components which may be used in a nucleic acid strand displacement reaction.

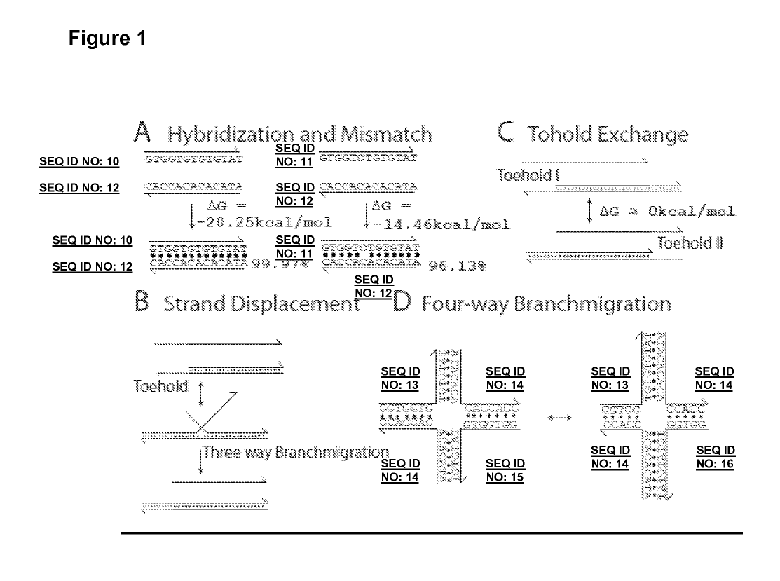

Strand displacement is a process through which two strands with partial or full complementarity hybridize to each other, displacing one or more prehybridized strands in the process. Precise and predictable binding between nucleic acid base pairs allows strands of different lengths to hybridize as shown in FIG. 1A. Energy gained from forming base pairs provides a driving force for hybridization. Substrates that are primarily double-stranded but have one or more single-stranded portions are used in many systems involving nucleic acid hybridization assays. The single-stranded regions, also known as toeholds, hold potential energy for hybridization and provide energy to drive reactions desired direction (FIG. 1B). There can be multiple toe-holds on one strand with different driving directions. The open (or unbound) and closed (or bound) states between toeholds can be exchanged through strand displacement. This exchange process is also known as toehold exchange (FIG. 1C). DNA strand displacement has been used to control logic and kinetics of DNA systems Zhang & Seelig 2011a.

Strand Displacement Systems and Methods for their Use

In some embodiments, the strand displacement systems described herein include a first catalyst molecule (also referred to as “catalyst strand,” “catalyst molecule” or “input molecule”). As described herein, a catalyst molecule is a single stranded nucleic acid molecule. In some embodiments, the catalyst molecule includes the nucleic acid sequence of a target biomarker of interest that, when detected, indicates the presence of a biologically significant or pathogenic process. In other embodiments, the catalyst molecule acts as an aptamer that binds to or is otherwise conjugated to an amino-acid based biomarker of interest (e.g., peptide, protein, antibody or fragment thereof) that, when detected, indicates the presence of a biologically significant or pathogenic process.

Biologically significant or pathogenic processes that may be indicated by the nucleic acid or amino acid-based biomarker include conditions or diseases that are associated with, for example, a gene mutation, one or more single nucleotide polymorphisms (SNPs), a specific strain of microorganism (e.g., viral, bacteria or fungal strains), a specific member of a family of closely related nucleic acid molecules (e.g., miRNA families), or expression of a particular protein or variant protein. In another aspect, an individual's response or predicted response to therapeutic intervention may be dependent on the presence or absence of the target biomarker. For example, in one embodiment, systems may be used to detect biomarkers associated with the M. tuberculosis complex and with resistance of tuberculosis (TB) to common antibiotics. In some aspects, the system may be used to diagnose TB antibiotic resistance to multiple drugs including rifampicin, isonizid, and kanamycin.

According to the embodiments described herein, the systems described herein may be used in methods for detecting a biomarker of interest. In addition, the systems and methods described below may be used to distinguish between molecules that differ by one or more nucleotides. Such methods may include a step of contacting the first catalyst molecule—which is present in or suspected of being present in a biological sample—with a nucleic acid gate molecule. Upon coming in contact with each other, the first nucleic acid catalyst binds the nucleic acid gate molecule.

In some embodiments, the nucleic acid gate molecule (also referred to herein as a “gate,” a “gate molecule” a “seesaw gate” or a “gate/sensor complex.”), is a double stranded nucleic acid molecule. The gate molecule may include a toehold strand and an output strand (also referred to herein as an “output molecule.”). In certain embodiments, a portion of the toehold strand includes a short nucleotide sequence called a “toehold sequence” or a toehold domain, which is complementary to a portion of the first catalyst molecule, called a toehold binding domain (together, “toehold domains”). In some aspects, the toehold binding domain is approximately 4-15 nucleotides in length, but may be any suitable length to accomplish strand displacement according to the embodiments described herein. The toehold binding domain is what allows binding between the first catalyst molecule and the gate molecule. Upon binding the toehold domain of the gate molecule, the first catalyst molecule initiates strand displacement of the output strand, releasing an output molecule from the gate molecule and forming a gate-catalyst complex.

The output molecule is a single stranded molecule that is released from the nucleic acid gate molecule through strand displacement that occurs when the first nucleic acid catalyst molecule binds to the nucleic acid gate molecule. As used herein, the output molecule may be referred to as the “output strand.” In certain embodiments, the release of the output molecule may result in the output molecule initiating strand displacement in a downstream reaction.

According to some embodiments, the output molecule produces a detectable signal upon release. The detectable signal may be any suitable signal sufficient to visualize or otherwise appreciate relative or definite amounts of the output molecule. In certain embodiments, the output molecule may be conjugated to a detection moiety, which, upon release of the output molecule, may emit a detectable signal. Such a detection moiety may include, but is not limited to, a colorimetric signal, a chemiluminescent signal (e.g., a fluorescent signal, a phosphofluorescent signal, or a luminescent signal), an electrochemiluminescent signal, or an electrochemical signal. In some embodiments, the detectable signal is a fluorescent signal. For example, in one embodiment, the nucleic acid gate molecule may include an output molecule that includes a fluorophore (e.g., FITC, Dig, GFP, YFP, RFP, xanthene derivatives such as fluorescein, rhodamine, eosin, Oregon green, Texas red; cyanine derivatives such as cyanine, indocarbocyanine, and thiacarbocyanine; naphthaleve derivatives; coumarin derivaives; oxadiazole derivatives; pyrene derivatives, oxaxine derivatives, arylmethine derivatives; and Tetrapyrrole derivatives) and a toehold strand that includes a quencher (e.g., using FRET or FET, a dexter electron transfer, chloride, iodide, acrylamide, rhodamine; or a dark quencher such as DABSYL, Qxt quenchers, Iowa black FQ or RQ, IRDye QC-1; or. In an intact gate molecule, the fluorophore of the output molecule is quenched by the toehold strand. Upon release of the output molecule, the fluorophore and quencher are separated, resulting in a detectable fluorescent signal by the output molecule. In another embodiment, the release of the output molecule results in the output molecule binding to a detection moiety or reporter complex, which results in a detectable signal. A reporter complex may be a double stranded nucleic acid and may contain a fluorophore and quencher located on separate nucleic acid strands. In one embodiment, the reporter complex contains one strand that is complimentary to a domain of the output molecule. Binding of the output molecule to the reporter complex results in strand displacement of one of the strands of the reporter complex, causing separation of stands containing the fluorophore and quencher, which produces a detectable signal. In other embodiments, the detection moiety may be a gold nanoparticle embedded with a nucleic acid or antibody that is complementary to the output molecule.

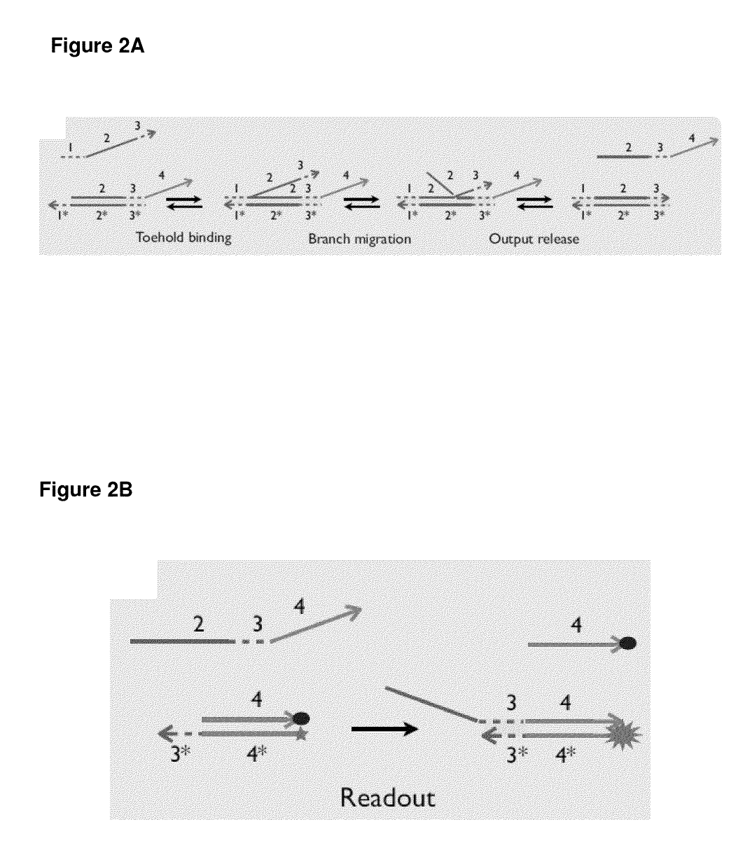

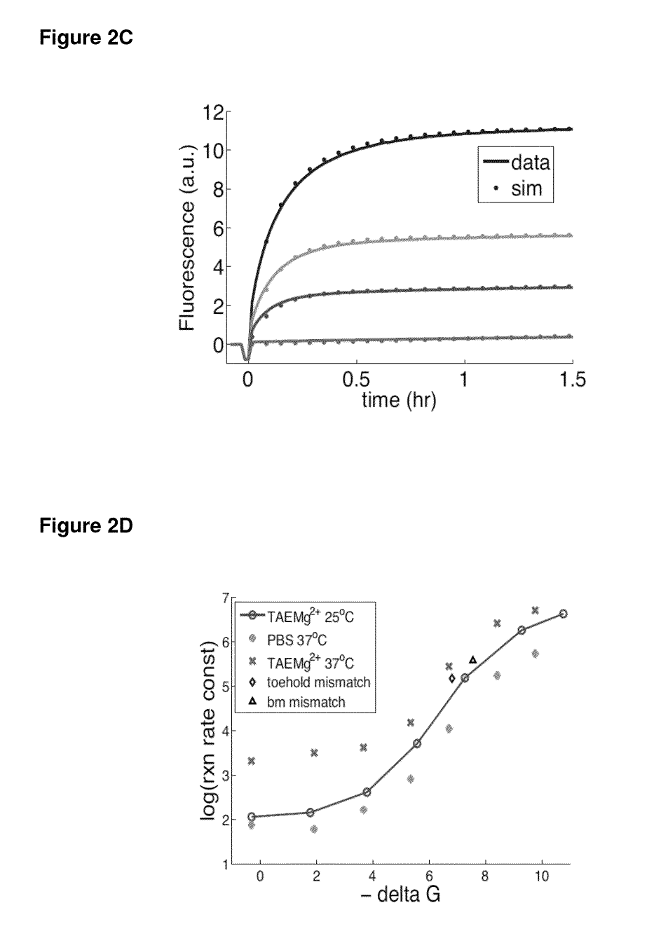

An example of a strand displacement reaction is shown in FIG. 2A. In this example, the functional portions or domains of each nucleic acid strand, are represented by numbers. A starred domain denotes a domain complementary in sequence to the domain without a star (e.g. domain 2* is complementary to domain 2) and complementary domains hybridize to each other via Watson-Crick base pairing (see FIG. 2A). The reaction is initiated when the two complementary toehold domains 1 and 1* bind to each other. In the subsequent random walk process, domain 2 of strand 2:3:4 competes with and is partially displaced by domain 2 of strand 1:2:3. The final step is the complete release of the initial binding partner, i.e. the separation of toehold domains 3 and 3*. The progress of strand displacement reactions is typically assayed using fluorescence (see FIG. 2B). Strand displacement releases at least one single-stranded nucleic acid product or output. In a DNA strand displacement cascade, this output serves as the input to a downstream reaction (see FIG. 2B). This mechanism enables autonomous signal propagation and thus makes it possible to connect individual components into multi-layered reaction networks (Seelig et al., 2006; Soloveichik et al., 2010).

It has been observed that the rate of strand displacement reactions can be quantitatively controlled over several orders of magnitude by varying the strength (length and sequence composition) of toeholds (see FIG. 2D) (Yurke et al., 2003; Li et al., 2002; Zhang et al., 2009). This feature enables engineering control over the kinetics of synthetic DNA devices.

Strand displacement cascades can also be used for catalytic signal amplification. Signal amplification is an important ingredient for many biosensing applications and is sometimes necessary for sensing low-concentration analytes. In one embodiment, strand displacement-based mechanisms are utilized for isothermal signal amplification.

Fuel Molecules Enhance or Amplify Signal Output of a Strand Displacement System.

In some embodiments, the system may include a nucleic acid fuel molecule that acts to bind and release the first nucleic acid catalyst from the gate-catalyst complex, allowing the first nucleic acid catalyst to be reused and initiate additional cycles of amplification. In this manner, a biological sample which has a very small quantity of the biomarker can be processed.

In a basic strand displacement reaction, the single-stranded catalyst or input is consumed in the course of the reaction, ending up in an inert double-stranded by-product (see FIG. 2A). In some embodiments, mechanisms may be used through which the same catalytic input molecule can participate in multiple strand displacement reaction cycles, thereby facilitating the release of many outputs and enabling signal amplification. The input can be thought of as acting catalytically, even if no covalent bonds are made or broken.

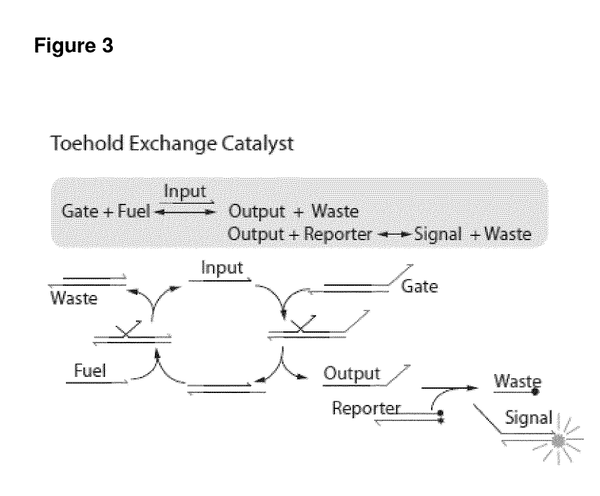

The reactants (other than the catalyst) of these non-covalent DNA catalysis systems generally include DNA strands or complexes that are kinetically trapped in metastable configurations, which act as “fuels” because they collectively store the energy that thermodynamically drives the catalyzed reaction forward (FIG. 3). Interaction between the catalyst and these fuels (via strand displacement) opens a fast pathway for the rearrangement of the fuels into products. The products can yield a fluorescence signal for detection, can lead to DNA nanostructure formation, or can be inputs for downstream strand displacement reactions.

This approach has been explored by demonstrating a system in which the hybridization of two complementary strands was slowed by constraining one or both of the strands via hybridization to shorter auxiliary strands (Tuberfield et al., 2003). A specific input strand could controllably reverse this constraint, and catalytically accelerate the formation of the double-stranded product. Increasingly sensitive and fast amplification mechanisms related to this approach have been demonstrated (Tuberfield et al., 2003; Bois et al., 2005; Green et al., 2006; Seelig et al., 2006; Zhang et al., 2007; Yin et al., 2008; Zhang et al., 2010; Qian et al., 2011).

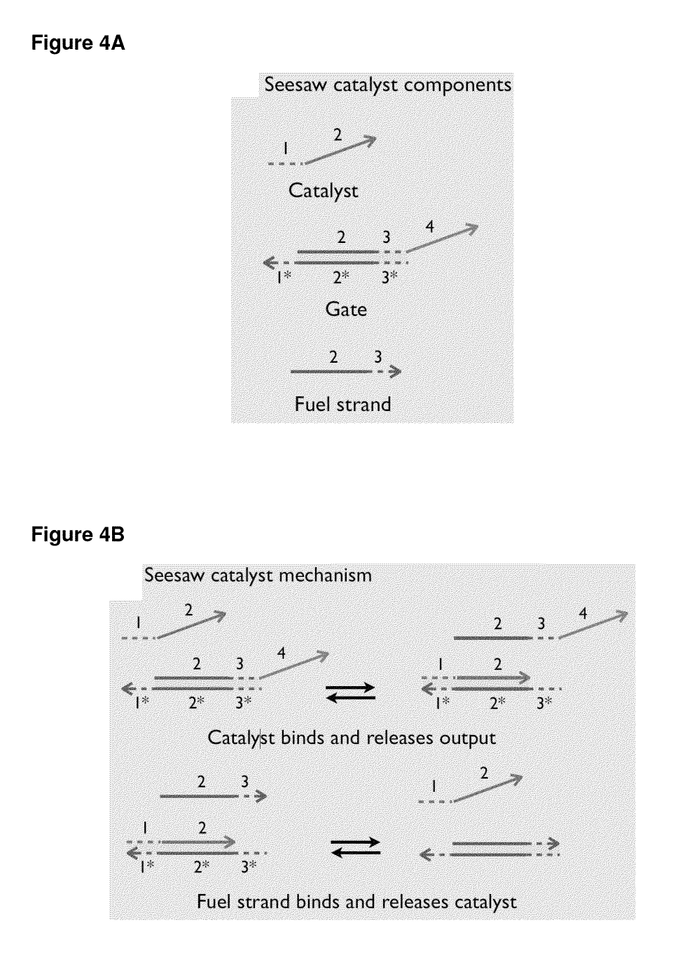

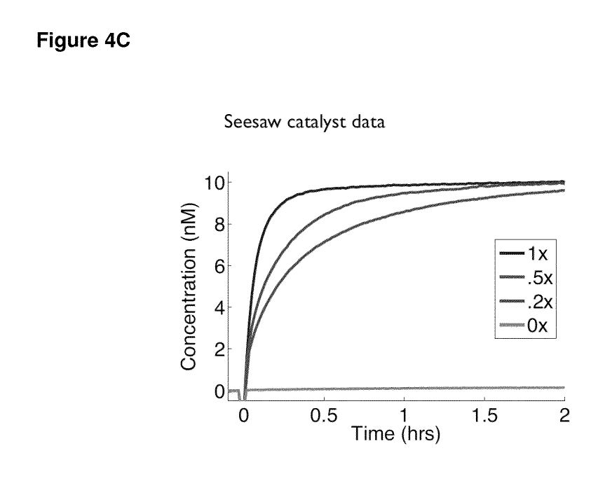

An example of a strand displacement system that includes a fuel molecule is shown in FIG. 4. This system includes a “catalyst” molecule and an “amplifier” to enhance or amplify the output signal (FIG. 4A). The amplifier includes two components, a double stranded “gate” complex and an auxiliary single strand, the “fuel” strand (FIG. 4A). The catalyst acts to initiate the interaction by binding the gate complex through a toehold interaction, which results in the catalytic release of an output molecule from each gate (FIG. 4B, top reaction). The fuel strand then binds and releases the catalyst, allowing the catalyst to interact with another gate molecule (FIG. 4B, bottom reaction). This cascade mechanism allows for the catalyst to sequentially interact with many gate complexes. Each reaction uses up one fuel strand and one gate complex (FIG. 4B).

Sink Molecules Enhance Specificity and Accuracy of Strand Displacement Systems

The systems detailed above describe strand displacement systems and methods for their use that detect differences or mismatches in nucleic acid strands by measuring the kinetics of strand displacement (FIGS. 2C and d, and FIG. 4C). However, one of the major limitations with measuring changes using kinetics is that it is sometimes difficult to reliably distinguish between catalytic molecules that differ by one or more nucleotides (a “mismatched catalyst strand”). This is because a hybridization-based probe will have a high affinity to both a perfectly complementary and mismatched strand. For example, although a mismatch of a single base normally causes about a 4 kcal/mol energy penalty, the energy gain from hybridization of forming other base pairs normally override the small penalty caused by the mismatch except at the melting temperature. In the example shown in FIG. 1A, the energy penalty of the mismatch is 5.79 kcal/mol. Under room temperature conditions, in a 1M sodium salt concentration with 1 uM of each single strand, 99.7% of the product would be a double stranded product without a mismatch and 96.13% of the product would have a mismatch. This small difference is hard to distinguish. To achieve high specificity, the reaction would need to be performed at melting temperature, which varies between sequences and experimental conditions.

Further, the difference between the signals produced by various catalysts may be observable during the early stages of amplification but not at the endpoint because all reactions eventually continue to completion. Additionally, it is difficult to distinguish an output signal for a target catalyst molecule from that of a mismatched catalyst strand without knowing the concentration of each, because the kinetics trace from a mismatch catalyst at a high concentration may appear similar to the kinetics trace from a target catalyst without a mismatch at a lower concentration. Furthermore, measuring reaction kinetics typically requires a sophisticated readout and an expensive instrument, while endpoint detection can be cheaper and can be accomplished much more simply. Thus, a system used to detect a biomarker of interest is needed that is capable of robustly distinguishing sequences that differ by one or more single nucleotide. Additionally, such a system should provide inexpensive and easy-to-use technology for testing in low resource settings.

Thus, in accordance with the embodiments described herein, the systems described above also include a nucleic acid sink molecule (also referred to herein as a “sink molecule”), which acts as a competitive gate molecule, or a targeted molecular “sink.” The sink molecule is a double stranded nucleic acid molecule that includes a competitive toehold strand that is fully complementary to a toehold binding domain of a putative second nucleic acid catalyst (also referred to as a “second catalyst,” or a “mismatched” strand or catalyst). In some embodiments, the putative second nucleic acid catalyst includes a nucleic acid molecule that is related to and/or similar to the first nucleic acid catalyst, but differs from the first nucleic acid catalyst molecule by at least one nucleotide. For example, the second nucleic acid catalyst molecule may be a mutant version of the first catalyst molecule that contains one or more SNPs, a different strain of a microorganism, an additional or alternative member of a nucleic acid family, or a nucleic acid aptamer that binds a mutated or alternate version of a protein or peptide biomarker. The nucleic acid sink molecule acts as a competitive binding substrate for the gate molecule because the second nucleic acid catalyst binds the sink with higher affinity than the gate. In this way, the sink acts to sequester the putative second nucleic acid catalyst as a result of the tight binding, thereby suppressing or preventing an unwanted output signal generated by the second catalyst molecule. According to embodiments described herein, a system that includes a sink molecule for detecting a biomarker of interest allows for robust discrimination of catalyst strands that differ by at least one nucleotide.

The methods may also include a step of sequestering a second nucleic acid catalyst molecule with a nucleic acid sink molecule, wherein the second nucleic acid catalyst differs from the first nucleic acid catalyst molecule by at least one nucleic acid. The method may also include a step of detecting the release of an output molecule.

Biomarkers of Interest

The systems and methods described above may be used to detect any wild type or variant biomarker of interest. In some embodiments, the target biomarker is a nucleic acid which includes, but is not limited to, DNA, such as genomic DNA, cellular DNA, acellular DNA, microorganismal DNA (e.g., bacterial DNA, viral DNA, fungal DNA, yeast DNA); and RNA biomarkers such as messenger RNA (mRNA), transfer RNA (tRNA), ribosomal RNA (rRNA) and ribozymes, viral RNA, small nuclear RNA (snRNA), microRNA (miRNA) and miRNA precursors, small interfering RNA (siRNA) and other regulatory RNA molecules (e.g., piRNA, IncRNA, etc.).

A nucleic acid biomarker may be single or double stranded. In one embodiment, molecular sensors and circuits may be built for the autonomous and embedded analysis of complex molecular mixtures. In some embodiments, these molecular sensors and circuits are used to detect and analyze single stranded catalysts or inputs such as wild type or variant miRNA or mRNA. Expression levels of microRNAs and messenger RNAs as well as deletions, insertions or mutations within messenger RNA sequences can be highly indicative of a disease state (see Sidransky et al., 2002; Weinberg et al., 2002; Calin et al., 2006; Esquela-Kerscher et al., 2006; Alvarez-Garcia et al., 2005 for examples of cancer markers, all of which are hereby incorporated by reference as if fully set forth herein). Circulating viral RNAs (Tsang et al., 2007, which is hereby incorporated by reference as if fully set forth herein) or microRNAs (Mitchell et al., 2008; Lawrie et al., 2008; Chen et al., 2008, which are hereby incorporated by reference as if fully set forth herein) found in blood are a promising class of biomarkers because they do not require significant processing from the sample. Further, miRNA have been reported to be highly stable in blood serum and the concentration of specific miRNAs can easily reach levels of about 100,000/μl in blood serum (Mitchell et al., 2008, which is hereby incorporated by reference as if fully set forth herein).

Double-stranded biomarkers may also be detected using the systems and methods described herein. For example, bacterial, viral, or cellular genomic DNA from various biological samples (e.g., blood, plasma, serum, urine, sputum, or other sample as described below) can serve as a catalyst molecule, but may require additional sample preparation. In particular, it may be necessary to separate the two strands in a duplex (e.g. by melting) such that one of two resulting single strands can serve as a catalyst or input to a circuit. This is similar to the processing required in hybridization-based DNA microarrays for the detection of genomic DNA.

Alternatively, mutations or other mismatched double-stranded DNA may be detected by four-way branch migration. Four-way branch migration has been observed in biology during meiosis crossover events. This process was previously modeled (Thompson et al. 1976) and branch migration speed has been characterized (Panyutin & Hsieh 1994). Four-way branch migration may therefore be used as part of a four-way toehold exchange mechanism, which is used to probe double-stranded DNA for SNPs that are important for many biomedical applications (Kim & Misra, 2007). See Example 2 below.

In other embodiments, the catalyst molecule is a nucleic acid aptamer, which acts as an adapter molecule to bind a target non-nucleic acid-based biomarker. In such embodiments, the target biomarker is an amino acid-based biomarker including, but not limited to, small peptides, peptide fragments, proteins, antibodies and antibody fragments (see Navani et al., 2006; Liu et al., 2009).

Strand Displacement Cascades May be Used to Build Multi-Component Circuits.

Strand displacement systems, such as those described herein provide a powerful mechanism for engineering multi-input circuits. Feed-forward digital logic circuits have been built that implement a complete set of logical functions (AND, OR, and NOT) using short oligonucleotides as inputs and outputs. Logical values “0” and “1” are represented by low and high concentrations, respectively. Because inputs and outputs are single-stranded nucleic acids, the gates may be cascaded to create multilayered circuits. Examples of nucleic acid-based logic gates, circuits that include nucleic acid-based logic gates and methods of performing operations with the gates and circuits are detailed in Seelig et al. 2006 and U.S. Pat. No. 7,745,594 to Seelig et al., the subject matter of both of which is hereby incorporated by reference as if fully set forth herein.

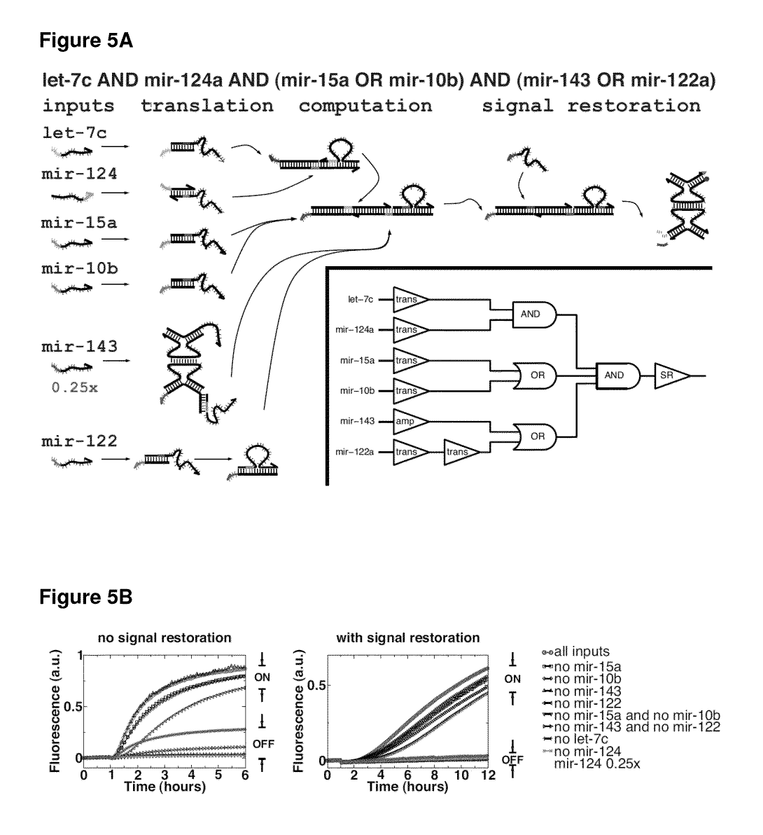

Logic gates work well with both DNA and RNA inputs because gate function relies solely on Watson-Crick complementarity. Additionally, these circuits operate well in the presence of potentially interfering biological RNA at a concentration in excess of gate concentration. Hybridization reactions provide the free energy to move computation forward and Watson-Crick base pairing between modular recognition domains determines the connectivity of gates. The scalability and modularity of the circuit has been shown by combining multiple components into circuits. The largest circuit takes 6 inputs and contains 12 gates in a cascade 5 layers deep (see FIG. 5).

These experiments show that a simple strand displacement reaction mechanism provides the basis for the construction of complex yet reliable molecular circuitry. The size of complexity of strand displacement-based circuits has been expanded (Qian et al., 2011). The DNA logic circuits discussed herein were designed for a situation where inputs can be represented as Boolean variables meaning that they are either present at a very high or very low concentration (Seelig et al., 2006; Qian et al., 2011). However, in many biological classification problems such a Boolean abstraction is an oversimplification. In recent work, it has been shown that it is possible to build DNA logic circuits that can sense and analyze information encoded in the concentrations of multiple analytes, even when not limited to the Boolean approximation (Soloveichik et al., 2010; Zhang et al., 2011).

Methods for Detecting a Biomarker of Interest Using a Clinically Relevant Diagnostic Tool.

In some embodiments, DNA-based biosensing circuits and systems, such as those described above, may be designed that can autonomously analyze and interpret the information encoded in a set of molecular disease markers. The use of molecular algorithms for biosensing is most promising in situations where advanced laboratory instrumentation is not available. Thus, in one embodiment, systems including synthetic molecular circuitry may be integrated with diagnostic tools (e.g., paper-based lateral flow devices) to generate easy-to-use diagnostic tests for low resource settings (LRSs). This approach allows multi-analyte testing in LRSs by providing simplified actionable readout of complex test results without the need for an instrument. As such, the systems and methods described above may be used for detecting a biomarker of interest in a biological sample. According to some embodiments, such methods may include a step of exposing a biological sample which contains or is suspected of containing a first nucleic acid catalyst molecule that includes a biomarker of interest to a diagnostic tool.

According to the embodiments described herein, biological samples that may contain or be suspected of containing the biomarker of interest include in vitro and in vivo biological samples. In vitro biological samples that may be used in accordance with the methods described herein may include, but is not limited to, cultured cells (e.g., cells with membrane-bound biomarkers or cultured cell lysates) or cell culture supernatant. In vivo biological samples that may be used in accordance with the methods described herein may include, but are not limited to, whole blood, serum, plasma, blood cells, urine, sputum, saliva, stool, spinal fluid or CSF, lymph fluid, the external secretions of the skin, respiratory, intestinal, and genitourinary tracts, tears, milk, neuronal tissue, lung tissue, any human organ or tissue, including any tumor or normal tissue, any sample obtained by lavage (for example of the bronchial system or of the breast ductal system), and also samples of ex vivo cell culture constituents. The sample can optionally be diluted with a suitable eluant before performing a diagnostic assay.

Thus, in one embodiment, the systems described above may be embedded in or otherwise part of a paper-based diagnostic tool, such as a lateral flow device. These devices may include a two-stage process of logic circuit reaction followed by target capture and detection. Paper networks were recently developed that include embedded timing mechanisms that allow programming of multi-step fluidic processes in simple paper devices. The concept arose from a history developing instrumented microfluidic cards for point-of-care diagnostics. For example, card-based assays were developed for detection of antigens (malaria) and IgM antibodies (measles, dengue, typhoid, Rickettsia) for differential diagnosis of infections that share the symptom of rapid-onset high fever (Yager et al., 2008; Yager et al., 2006). The assays were carried out on a nitrocellulose membranes using dry gold detection reagents stored on-card. The purpose of the card and instrument was to carry out the processing

In some embodiments, the biological sample may be exposed to the diagnostic device without additional processing. However, in some embodiments, the biological sample may undergo further processing to isolate, enrich, and/or amplify the nucleic acid in the sample. Such processing methods are known in the art and are commercially available (e.g., Qiagen, Sigma Aldrich, Promega, Invitrogen, Norgen Biotek Corp,), and include, but are not limited to, cell lysis, column-based purification, ethanol precipitation, phenol-chloroform extraction, Trizol extraction.

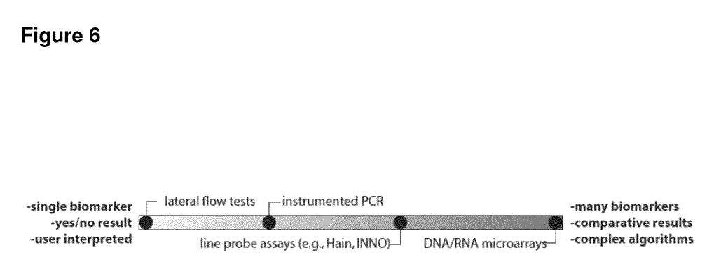

Suitable diagnostic tools that may be used according to the methods described herein include, but are not limited to, microfluidic devices, lateral flow tests (LFTs), instrumented PCR methods, line probe assays (e.g., Hain, INNO), and nucleic acid microarrays. These diagnostic tools vary from very simple tests appropriate for LRSs through very complex tests which are more suited for lab settings (see FIG. 6). Nearly all laboratory assays are based on multiple processing and detection steps carried out in a timed sequence by a technician or a machine. For example, PCR normally requires sample preparation by a trained operator followed by instrumented amplification and detection by a fluorescence reader. The enzyme-linked immunosorbent assay (ELISA) is widely used for detection of proteins, antibodies, or some small molecules; it requires a dozen or more manual steps with timed incubations followed by absorbance measurement by a dedicated instrument. Tests like these are useful when time and facilities are abundant, but not in field or clinical settings where easy-to-use rapid turnaround analytical tests are desired (e.g., infectious disease diagnostics, detection of biowarfare agents, water quality testing) (Peeling et al., 2010; Urdea et al., 2006; Yager et al., 2008; Yager et al., 2006).

According to some embodiments, the diagnostic tool uses a paper-based diagnostic tool that includes a reaction with a visual readout, such as paper-based a microfluidic device or a lateral flow test (LFT) (also referred to as a “lateral flow device”). An LFT diagnostic tool is based on the wicking of a sample through a matrix or other interface, treated with biochemical reagents (e.g., pregnancy tests are LFTs) (Shih et al., 2010). LFTs and other paper-based diagnostic tools provide rapid test results (5-30 minutes), can be run by an untrained user, and allow visual detection of the test result (e.g., pregnancy test lines are gold nanoparticles or latex beads). An LFT diagnostic tool may comprise any suitable material able to wick fluids by capillary action including, but not limited to, paper (e.g., filter paper, chromatography paper), cellulose, cellulose acetate, nitrocellulose, cloth, or a porous polymer film. Nitrocellulose is a widely used paper in LFTs; it is a notable material as a reaction substrate (O'Farrell et al., 2009).

Although LFTs and other paper-based diagnostic tools are typically created from a straight paper strip and used to detect one catalyst molecule, it is appreciated that the LFTs and other paper-based diagnostic tools described herein may include different shaped (e.g., tree-shaped) paper devices that split a sample into discrete zones with different test chemistries (Martinez et al., 2007; Martinez et al., 2008; Dungchai et al., 2010; Li et al., 2010; Wang et al., 2010; Zhao et al., 2008.) or may include more than one reaction and/or detection on a single strip of paper such that each reaction proceeds in series. Such paper devices provide an advantage over conventional LFTs by allowing multiple tests to be performed on a single sample, however they do not aid the user in interpreting the results of multiple tests. Therefore, a test that aids the user in interpreting the results of multiple tests would be useful.

Therefore, in some embodiments, an LFT or other paper-based diagnostic tool includes at least one reaction zone and at least one detection zone, and may include a plurality of reaction and detection zones to visually detect the one or more results of a single or multiple test. The reaction and detection zones contain one or more components of a strand displacement system, such as those described in detail above. In certain embodiments, a reaction zone includes a set of reaction components, which include at least one gate molecule and at least one sink molecule (described in detail above), both of which are attached to the LFT substrate directly (e.g., by drying a solution of reaction components onto the paper matrix) or indirectly (e.g., via beads within the fiber matrix of a paper-based LFT). In certain embodiments, the set of reaction components also includes at least one fuel molecule. When a biological sample that contains or is suspected of containing a biomarker of interest (i.e., a first catalyst molecule) and/or a second (or “mismatched”) catalyst molecule is exposed to a corresponding reaction zone, the first catalyst molecule binds the gate molecule, initiating a strand displacement reaction and releasing an output molecule, as described above. Any suspected mismatched catalyst molecules that are present in the biological sample bind preferentially to the sink molecules, thereby quenching mismatched molecules and preventing a signal output attributable to mismatched molecules.

In some embodiments, the LFT also includes a time delay zone. The time delay may include one or more sugar solutions dried on the LFT that create fluidic time delays that can be controlled by the sugar concentration.

The methods described herein may also include a step of detecting the biomarker of interest by visualizing a change in signal when the output molecule binds a nucleic capture molecule that is attached to a detection zone. In certain embodiments, the one or more detection zone includes output capture probes, which are attached to the LFT directly or indirectly. The output capture probes are complementary to the output strands released in the reaction zone, such that the output strands hybridize to the output capture probes, thereby immobilizing or capturing the output strands within the detection zone.

As described above, the output strand emits an output signal which is visually detectable on an LFT at the detection zone. In some embodiments, the output strand, when released from the gate molecule, emits a fluorescent signal that represents presence of the first catalyst molecule (i.e., the biomarker of interest). Any suitable detection moiety may be conjugated to the output strand, as described in detail above. Alternatively a detection moiety (e.g., gold nanoparticles) may be conjugated to the output capture probe or directly attached to the detection zone of the LFT.

LFTs (or nucleic acid lateral flow, NALF) have been used for visual detection of DNA from culture or DNA that has been amplified in a benchtop instrument (Ngom et al., 2010). Thus, in some embodiments, the output strand may be captured and the output signal visually detected by any method known in the art including, but not limited to, amplicon hybridization to immobilized probes (Edwards et al., 2006; Kalogianni et al., 2011; Corstjens et al., 2003; Carter et al., 2007; Mao et al., 2009; Rule et al., 1996, the subject matter of all of which is hereby incorporated by reference herein, as if fully set forth herein), antibody binding to hapten-tagged amplicons (antibody-dependent) (Kalogianni et al., 2011; Corstjens et al., 2001; Noguera et al., 2011, the subject matter of all of which is hereby incorporated by reference herein, as if fully set forth herein), and streptavidin binding to biotin-tagged amplicons (Corstjens et al., 2003; Corstjens et al., 2001; Noguera et al., 2011, the subject matter of all of which is hereby incorporated by reference herein, as if fully set forth herein). In some embodiments, visible labels, including gold nanoparticles, may be used to detect amplified products. Line probe assays (LiPAs) represent one example of extending this format to parallel detection of multiple markers; e.g., the Hain Genotype MTBDR+ test includes 24 readout lines to identify TB strains and common resistance genes (Abebe et al., 2010; Bang et al., 2006; Brossier et al., 2006; Cavusoglu et al., 2006; Friedrich et al., 2011; Hillemann et al., 2005; Hillemann et al., 2005; Miotto et al., 2006; Parsons et al., 2011). Although LiPAs make it possible to ask complex diagnostic questions, they require careful interpretation by an expert. In one embodiment, a novel diagnostic approach embeds interpretation algorithms in a molecular circuit to provide a simple actionable readout from complex test results. This approach may be applicable to a wide array of analytes and multi-analyte testing algorithms.

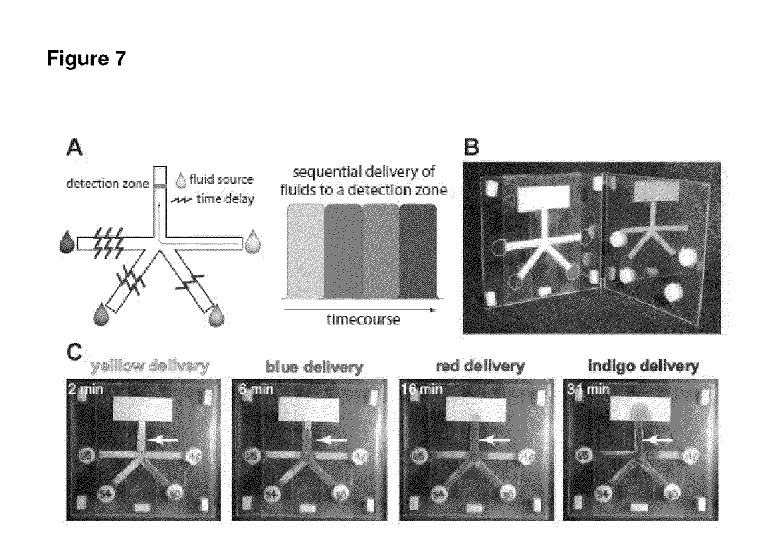

As described above, LFTs are an excellent candidate for point of care diagnostics—they are fast, easy to use, and do not require an instrument. Paper-based LFTs materials have also incorporated automatic volume metering and timing mechanisms based on shaped paper networks (Fu and Lutz et al., 2010; Fu and Ramsey et al., 2010; Kauffman et al., 2010; Osborn et al., 2010), such that assays that require multiple timed steps can be programmed into inexpensive paper devices (see FIG. 7 for one example).

It is widely recognized that eliminating the need for an instrument is an important step to reach settings outside the laboratory, since instruments are not only expensive, but they are more likely to break and less likely to be serviceable (Peeling et al., 2010; Urdea et al., 2006; Yager et al., 2006; Yager et al., 2008). In conventional devices, removing the instrument shifts more burden onto the user because they must perform more steps and must interpret the results, which directly leads to increased potential for user error. The combination of DNA logic circuits and systems with low-cost paper-based diagnostic devices provides a solution to these outstanding challenges in point of care diagnostics.

The following examples are intended to illustrate various embodiments of the invention. As such, the specific embodiments discussed are not to be construed as limitations on the scope of the invention. For example, one of skill in the art will appreciate that the system described herein represents a platform technology and, as such, specific sequences of the system components described herein may be tailored to any chosen biomarker. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLES

### Example 1

### DNA Amplifiers with Single Nucleotide Specificity

An amplification system has been developed that can discriminate between single strands of nucleic acid that differ at one or more single nucleotide position. This system may contain a strand without a nucleic acid base mismatch (called a “catalyst”), which can trigger an amplification reaction leading to a strong output signal, while a strand with a single nucleotide difference (called a “mm-catalyst”) does not lead to any discernible signal at the end of the experiment. Distinguishing single-stranded nucleic acids that differ by only a single position is a challenging problem because any hybridization-based probe will have a high affinity to both the perfectly complementary strand and the mismatched strand. The experiments performed in this Example may be performed in solution phase and may be used reliably at room temperature.

Using this system, one DNA or RNA strand containing a specific known mutation may be distinguished from a sequence that does not contain this mutation. Amplification of the target over the mutated sequence using this system may be at least approximately a 100-fold to at least approximately a 1000-fold amplification. This approach may be extended to the more difficult problem of selectively amplifying one or multiple sequences from a family of closely related sequences. For example, the let-7 family of microRNAs may be used as a biologically relevant sample (Roush et al., 2008) as described in more detail below. There are on the order of 10 let-7 family members in humans that differ from each other by only one or two mutations. The let-7 family thus provides an ideal test case for this technology. The assays used are fluorescence-based kinetics experiments (see FIG. 2 for typical reaction conditions).

Results.

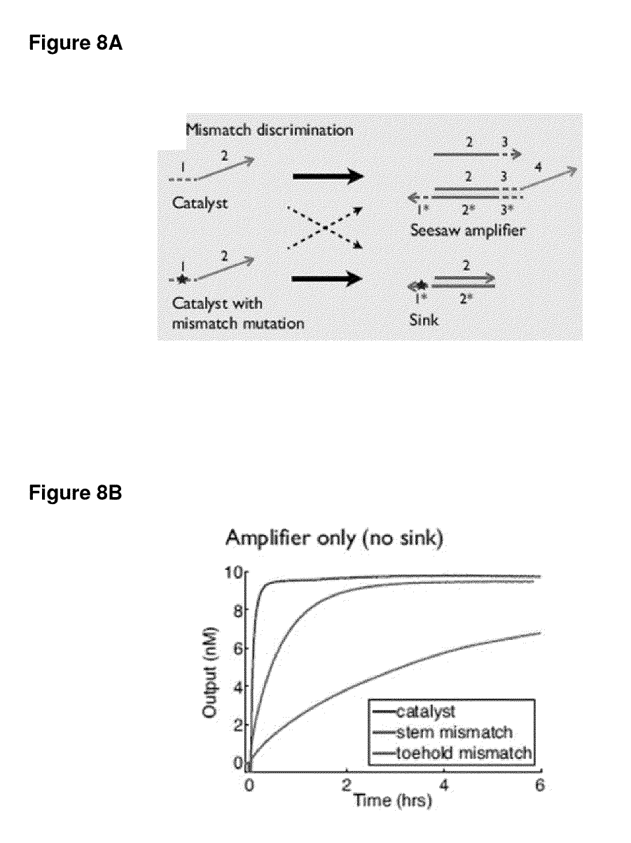

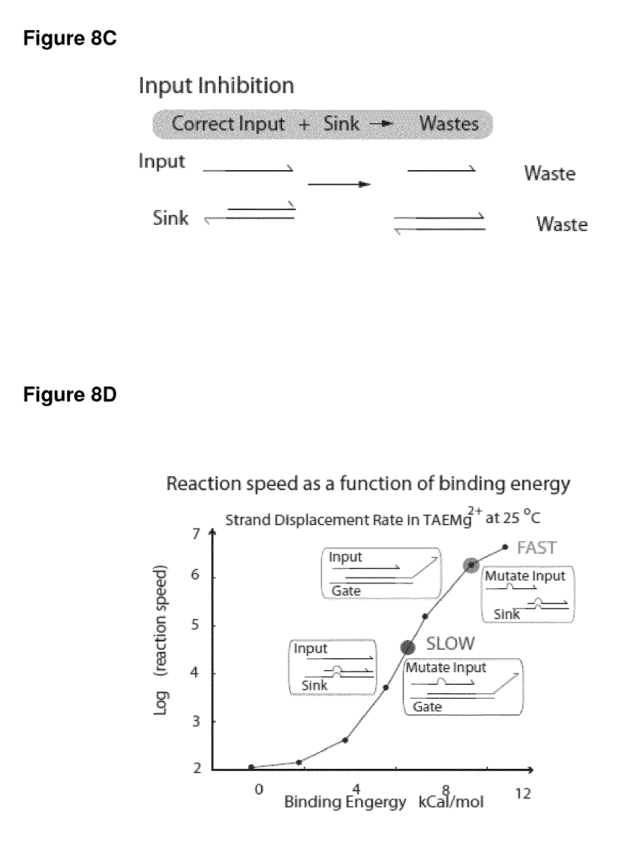

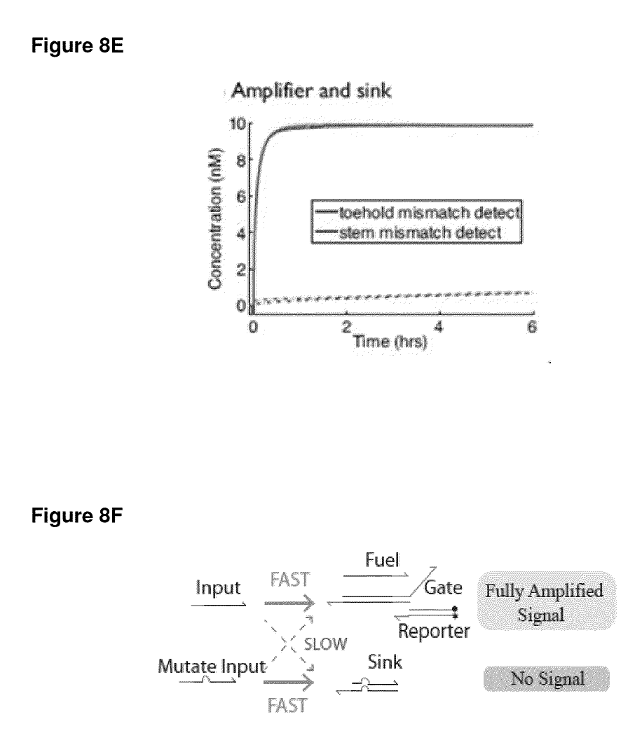

A first mismatch discrimination module is based on the “seesaw” amplifier system (Qian et al, 2011; FIG. 4A; FIG. 8A catalyst and seesaw amplifier only). The kinetics of strand displacement reactions depend strongly on the strength (length and sequence composition) of the toehold, which is an important aspect for this system. Shortening the toehold domain of the input strand (domain 1 in FIG. 2A) by a single base can decrease the reaction rate constant up to about 10-fold; introducing a mismatch in the same toehold domain produces a similar effect (FIG. 2D). (Introducing mismatches in the branch migration domain 2 also leads to observable differences (FIG. 2D); here, the focus is on toehold mismatches). Mismatch discrimination using a single strand displacement reaction was reported previously (Li et al, 2002); however, in this experiment, mismatch discrimination is integrated with amplification.

The experimental data in FIG. 8B shows that a simple seesaw catalytic system is highly sensitive to a single mismatch in the toehold domain of the catalyst strand. However, although reaction kinetics are slowed down considerably relative to a system that contains no mismatch in the input, the difference is only observable in the early stages of amplification and not at the endpoint: both the catalyst and the mm-catalyst eventually turn over all available substrates (fuel+gate) and thus produce the same endpoint signal. Therefore, although the speed of the reaction (strand displacement kinetics) is very sensitive to the change in binding energy, endpoint discrimination is not achieved in this catalytic reaction because the reaction only proceeds in one direction. All correct and mismatched catalysts trigger the reaction, although at different rates, and eventually all go to completion. Thus, it is difficult to distinguish differences between multiple inputs by looking only to the endpoint signal.

Discrimination based on reaction kinetics is unsatisfactory for two reasons. First, knowledge of catalyst concentrations is required. For example, both catalyst and mm-catalyst strands are used at the same concentration in the experiment presented in FIG. 8B. However, increasing the concentration of the mm-catalyst in the reaction could make the corresponding trace look similar to the trace for the catalyst without a mismatch. Consequently, the reaction rate constant will not be accurate unless the concentration of all reactants is known. Second, measuring reaction kinetics requires a sophisticated readout and an expensive instrument, while endpoint detection can be cheaper and accomplished much more simply.

To achieve endpoint discrimination, different destinations were provided for different inputs in an amplification system by adding competitive binding substrates. The system shown in FIG. 8A combines a seesaw catalyst with a competitive threshold gate or “sink.” The sink is a strand displacement gate where the longer strand is fully complementary to the mm-catalyst (FIG. 8C). The mm-catalyst binds with the sink approximately 100 times faster or more than with the seesaw gate; thus, the mm-catalyst is more likely to bind to the sink than it is to interact with the seesaw gate (FIG. 8D). Furthermore, binding to the sink is essentially irreversible while binding to the seesaw gate is reversible by design. Conversely, unlike the mm-catalyst, the catalyst is about 100 times more likely to bind to the seesaw gate. The overall effect is that the mm-catalyst is rapidly bound by the sink and does not produce a signal, while the catalyst binds the seesaw gate, which triggers a catalytic amplification cycle leading to a strong signal (FIG. 8E). By using different gates and sinks, different inputs can “choose” their own path and achieve different endpoint states (FIG. 8F). This reaction mechanism may be further characterized for a wide range of concentrations of all components. Additionally, RNA may also serve as an input in this system.

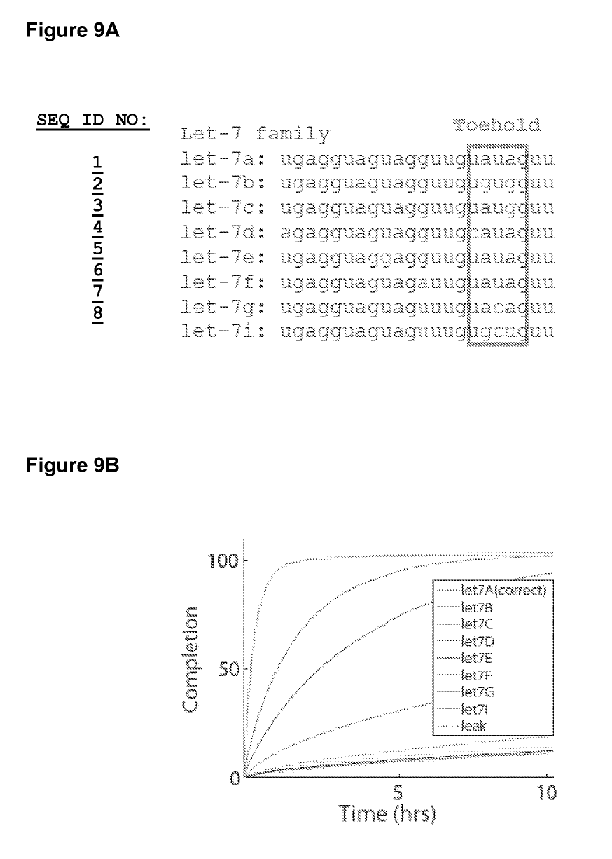

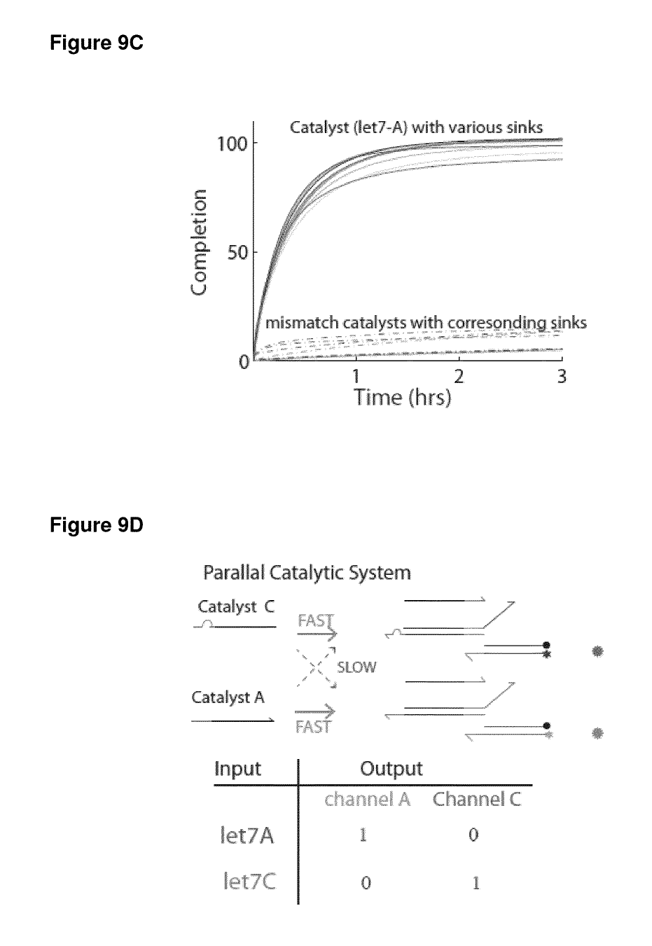

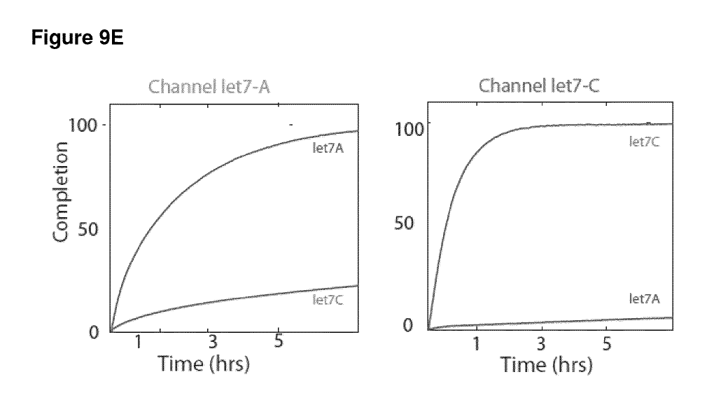

As a further application of this mechanism, an amplification system for the detection of the Lethal-7 (let-7) microRNA family was designed (FIG. 9). The let-7 family is a group of miRNAs with very similar sequences that play an important role in human cancer and can be used to diagnose cancer cells. Eight similar sequences from the let-7 family were tested as shown in FIG. 9A. These sequences are shown below (bolded nucleotides show differences between family members and underlined nucleotides represent toeholds):

(SEQ ID NO: 1)
let-7a: UGAGGUAGUAGGUUGUAUAGUU
(SEQ ID NO: 2)
let-7b: UGAGGUAGUAGGUUGUGUGGUU
(SEQ ID NO: 3)
let-7c: UGAGGUAGUAGGUUGUAUGGUU
(SEQ ID NO: 4)
let-7d: AGAGGUAGUAGGUUGCAUAGUU
(SEQ ID NO: 5)
let-7e: UGAGGUAGGAGGUUGUAUAGUU
(SEQ ID NO: 6)
let-7f: UGAGGUAGUAGAUUGUAUAGUU
(SEQ ID NO: 7)
let-7g: UGAGGUAGUAGUUUGUACAGUU
(SEQ ID NO: 8)
let-7i: UGAGGUAGUAGUUUGUGCUGUU

Because DNA is cheaper and more stable than RNA, the system was tested using DNA inputs of the same sequences of the let-7 miRNAs. A system was first built for the explicit detection of let-7A; however, the system can also be triggered by other members of the let-7 family (FIG. 9B). Additionally, when corresponding sinks were added to the system reaction, the reactions containing mismatched inputs produced little signal (FIG. 9C, mismatch catalysts with corresponding sinks) while reactions containing the correct input continued to completion as if there were no sink present (FIG. 9C, catalyst (let7-A) with various sinks). Due to the similarities of DNA with RNA, this system may also be directly tested utilizing an RNA input.

Different paths for different sequences can also be set using a gate instead of a sink. Gates for mismatched inputs can serve as sinks for the correct catalyst. Two catalytic systems were designed for let-7A and let-7C, which contain only one base difference (FIG. 7D). When let-7A was added to the reaction, a fully triggered signal was produced in channel A (FIG. 7E, left panel, blue line) while no signal was produced in channel C (FIG. 7E, right panel, blue line). Whereas, when let-C was added to the reaction, no signal was produced in channel A (FIG. 7E, left panel, green line) while a fully triggered signal was produced in channel A (FIG. 7E, right panel, green line).

Furthermore, there is the potential to generate higher discrimination factors by taking advantage of this catalytic system in which input can be reused. This system may also be built on paper based lateral flow devices (Fu et al., 2010) to make high specificity diagnostic devices that can be used easily everywhere.

It is desirable to increase the sensitivity of strand-displacement based amplifiers to the level that is sufficient for direct detection of biomarkers from biological samples without additional amplification. For example, a two-stage catalytic amplifier cascade that was sensitive to a concentration of 1 pM (or 600,000 molecules/μl) of a synthetic input was previously reported (Zhang et al., 2007). For comparison, another report demonstrated that the concentrations of diagnostic miRNA biomarkers in blood are in the range of 10,000/μl to 100,000/μl (Mitchell et al., 2008). This implies that strand displacement-based amplification circuits are sensitive to concentrations in the range relevant for biosensing applications. The sensitivity also depends strongly on the type of measurement and conditions of the readout. However, it would be desirable to increase sensitivity by at least an order of magnitude.

Gel-based purification may be used for assembly of the amplifier complex in addition to other methods of purification of nucleic acids to enhance the sensitivity. Further, restriction enzymes may be used to process gate components from biological DNA (thus starting from an essentially perfect template) and ligation may be used to assemble long DNA strands from multiple short pieces that can be synthesized with high fidelity.

In addition, modified bases such as 2′O-methyl RNA bases and Locked Nucleic Acid bases may be used as components of nucleic acid amplifiers. Introduction of modified bases may be used to control the thermodynamics and kinetics of reactions involving multi-stranded complexes. Such modifications may help to decrease leak reactions by improving the stability of base pairing at nicks or at helix ends. To increase gain even further, multi-step cascades of fixed gain amplification reactions may be designed. Such cascading enables large gain, even if the gain in each stage is limited.

### Example 2

### Double-Stranded Mutation Detection

Detection of double-stranded DNA is difficult because the DNA is already in a minimum energy state and does not have any reactive single-stranded bases available for binding. In typical experimental conditions (room temperature, non-denature), the time for a double-stranded molecule to spontaneously dissociate can be months to years. Thus, to detect mutations in double-stranded DNA, researchers normally separate the double-stranded DNA into two single-strand molecules by melting the DNA. Various methods have been used to get pure single-stranded DNA from genome. However, long single-stranded nucleic acids often adopt complex secondary structures, in which regions are inhibited from hybridization to the detection probe. To avoid secondary structure, it is desirable to keep the target double-stranded. To do so, a double stranded probe may be generated to take advantage of four-way branch migration. By avoiding secondary structure of dissociated strands, there is no limitation on target sequence. Thus, longer and more efficient probes may be more easily designed.

As a further advantage, an improved discrimination may be obtained using double-stranded probes. Because mutations in double-stranded molecule happen on both sides, there are two mismatched nucleotides in the target double stranded molecule. As such, the resulting energy penalty is doubled compare to a single-stranded detection system.

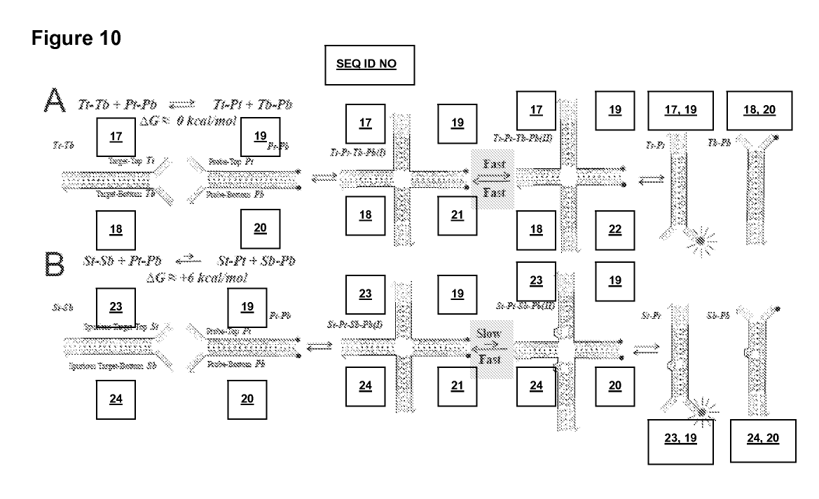

The reaction mechanism is explained in FIG. 10. With the complementary ‘fork’ toehold on Probe and Target (orange and purple), a Target-Top (Tt) strand hybridizes with a Probe-Top (Pt) strand; and a Target-Bottom (Tb) strand hybridizes with a Probe-Bottom (Pb) strand. This toehold binding event initiates a four-way branch migration. Because the four-way branch migration region has homologous sequences at the junction point (called Holliday junction), each nucleotide on Tt has the same probability of binding its complement on Tb or Pt.

Because each branch migration step has the same probability of proceeding forward and backward, the junction will ultimately reach the toehold (blue and green) on the opposite end. Spontaneous dissociation of the blue and green toehold results in the product TtPt and TbPb, each with exposed (or “open”) blue and green toeholds. In addition, the fluorophore and quencher interaction are broken, allowing a fluorophore to be detected. The exposed blue and green toeholds on the products may initiate reverse reaction and drive the product to the original state. The strength of all toeholds may be adjusted, such that the forward and backward reactions have similar speed. Fast reaction speed on both directions ensures rapid approach to equilibrium. Using this design, the hybridization yield (X) is calculated as follows:

$$χ = \frac{[ TtPt ]}{{[ Probe ]}_{time = 0}} \approx 0.5$$

In the case of a spurious target (FIG. 10B), once the branch migration reaches the mismatched point, the mutated nucleotide on the Spurious-Top (St) strand is much more likely to bind to the Spurious-Bottom (Sb) strand than to the Probe-Top (Pt) strand, because Sb contains the same mutation that complimentary to the mutation on St. If St binds to Pt, the mutated base will not hybridize with the base on Pt. The formation of each mismatch typically carries an energy penalty approximately 4 kcal/mol. Also, Sb will bind to Pb and has similar energy penalty. Therefore, the binding of the spurious target results in a total free energy that is approximately 8 kcal/mol less favorable than the perfect match. This large energy penalty makes the forward reaction much less favorable than the reverse reaction. As such, few products (StPt and SbPb) are made, such that X<<0.5.

The discrimination factor, Q, is defined as the hybridization yield ratio of the correct target over the mutated target when they have same concentration as the probe:

$$Q = \frac{{χ}_{correct}}{{χ}_{mutated}}$$

Additionally, the concentration tolerance, R, is defined as the ratio of concentrations needed to achieve 50% yield at equilibrium:

$$R = \frac{{[ StSb ]}_{χ = 0.5}}{{[ TtTb ]}_{χ = 0.5}}$$

A large R value ensures specific discrimination over a large range of concentrations.

According to some embodiments, a model can be build to predict sensitivity. The reaction equation of the model may be written as:

PtPb+TtTb [symbol] PtTt+PbTb

and has initial condition:

PtTt=PbTb=0,PtPb=c,TtTb=n·c

At equilibrium, the initial condition is represented as follows:

PtT\hat{t}=PbTb=x,PtPb=c−x,TtTb=n·c−x.

Thus the equilibrium constant is reflected in the following equation:

$${K}_{eq} = \frac{{x}^{2}}{( c - x ) ( nc - x )}$$

If the hybridization yield is defined as X=x/c, the above equation can be written as:

$${K}_{eq} = \frac{{χ}^{2}}{( 1 - χ ) ( n - χ )} \\ χ = \frac{{K}_{eq} + {K}_{eq} \cdot n - \sqrt{{K}_{eq}} \sqrt{{{K}_{eq} ( n - 1 )}^{2} + 4 n}}{2 ( {K}_{eq} - 1 )} \qquad ( Equation 1 )$$

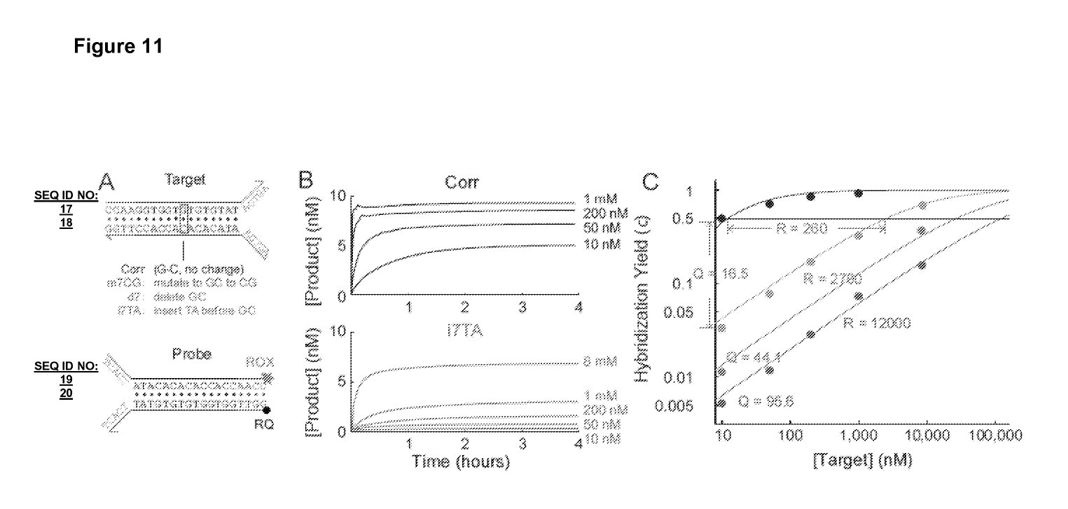

Because K_{eq}=Exp(−ΔG/RT), where ΔG is the free energy of the reaction, R=1.986 cal/mol/K is the gas constant and T=298K is the room temperature. Thus, hybridization yield X can be expressed as a function of the ΔG and n (input concentration), as shown in FIG. 11C (curve). From Equation 1, when X is small, then:

x≈\sqrt{K_{eq}·n},

which means that the hybridization yield grows as a square root of input concentration, or, in other words, R≈Q² (Equation 2).

Thus, this system should have quadratic tolerance on concentration. To test our prediction, experiment to test a high concentration of mutated target was performed. Using Equation 2 (R≈Q²), the experimental results agreed with the relationship, as shown in FIG. 11.

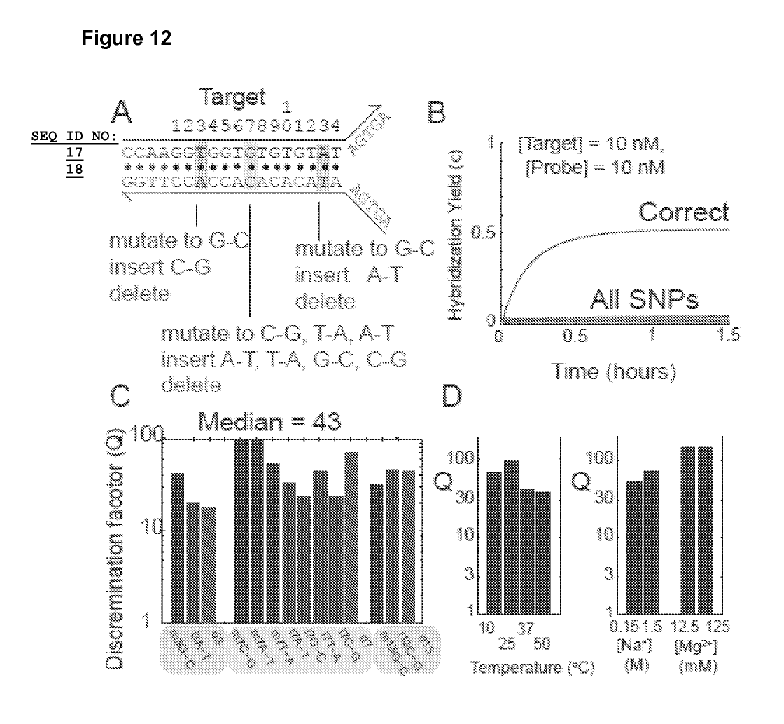

This reaction TtTb+PtPb [symbol] TtPt+TbPb is a bimolecular reaction with two products and has no net gain in base pair binding (ΔH=0), no enthalpy change, a very small AG change, thereby ensuring that the system is robust under varied temperatures, concentrations, and salinity. In an experimental study, this system is also robust under conditions of 10° C., 37° C., 50° C., and in Na+ or Mg2+ solution of various concentration. Various mutations with different positions and identities were tested, and produced a median Q of 43 as shown in FIG. 12.

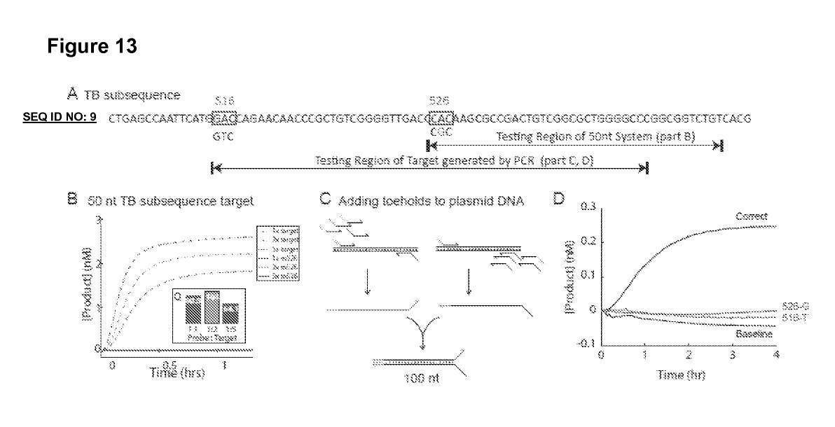

To test robustness over sequence length, experiments were performed on a TB subsequence that is 50 nt in length (CTGAGCCAATTCATGGACCAGAACACCCGCTGTCGGGGTTGACCCACAAGCGCC GACTGTCGGCGCTGGGGCCCGGCGGTCTGTCACG (SEQ ID NO:9)) (FIG. 13A), resulting in a Q≈100 (FIG. 13B). To illustrate a typical detection experiment, detection of a drug resistant marker on tuberculosis (TB) was used. A TB subsequence (100 base pairs in length) was cloned into a plasmid. Besides the wild type, a plasmid with a TB sequence having a mutation at amino acid positions 516 and 526 (FIG. 13A). Experiments were performed to detect the single base differences between three plasmids. The ‘fork’ toeholds may be generated using unbalanced PCR followed by annealing as shown in FIG. 13C. The annealed product was tested and purified using grouse gel and tested with a probe (ordered from IDT, pre-annealed). The kinetics results are shown in FIG. 13D, illustrating that the plasmid with wild type TB sequence was easily distinguished from the mutated TB sequence. This system shows robustness with an unknown concentration and salinity under room temperature and can be used to detect mutation for a random sequence around 100 base pairs.

### Example 3

### Multi-Input DNA Logic Circuits for Diagnostic Applications

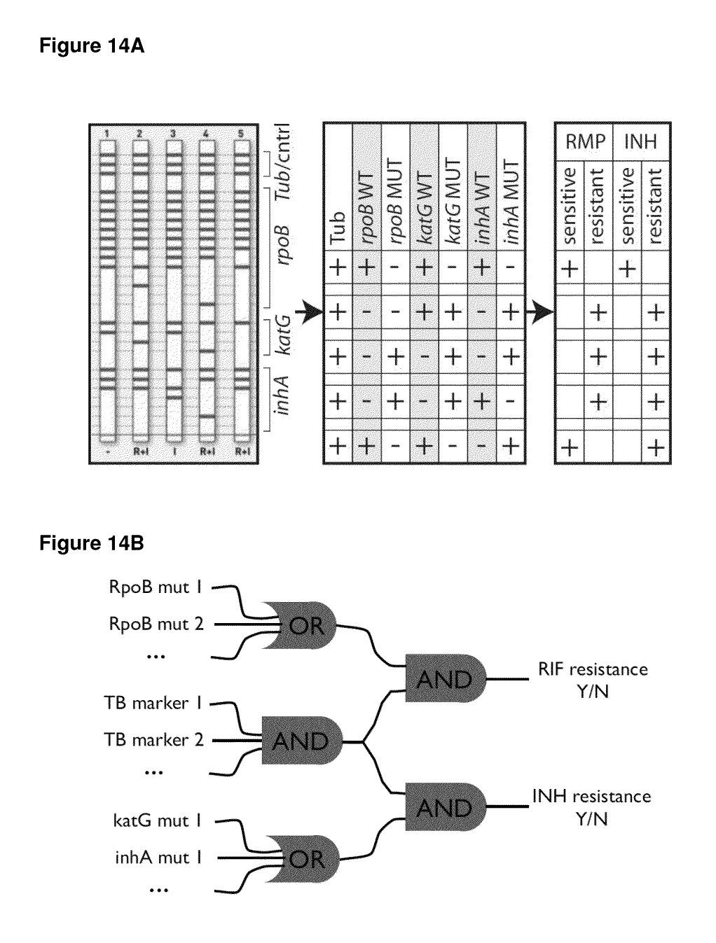

Several amplifier modules may be integrated into a molecular circuit for multi-input analysis. For example, molecular markers associated with the M. tuberculosis complex and with resistance of tuberculosis (TB) to common antibiotics may be detected and analyzed using multi-input DNA logic circuits as described herein. The design may be based on established molecular markers and sequences used in line probe assays (e.g., Hain Genotype MTBDR+, INNO-LiPA TB). However, the complexity of the readout and thus of the diagnostic challenge can be drastically reduced using molecular logic. Instead of reading out each marker independently as is currently done in line probe assays (FIG. 14A, left and center panel), a DNA circuit may be used to analyze the information encoded in these markers and provide a signal corresponding to a diagnostic outcome of interest (FIG. 14B). In these experiments, synthetic single stranded DNA and RNA sequences may be used as inputs. After the experiment is optimized, actual TB samples may be tested using this method. The experiment performed in this Example may be performed in solution phase.

The ability to identify single point mutations is important for diagnosing antibiotic resistance in TB. TB is classified as multi-drug resistant (MDR) if it is resistant to the first-line antibiotics rifampicin (RIF) and isonizid (INH). Extensively drug resistant (XDR) TB is further resistant to second-line antibiotics such as kanamycin. Most of these drug resistances can be traced to individual point mutations in narrowly defined regions within a few genes. For example, mutations in an 81 base pair region in the rpoB gene are responsible for 95% of the observed cases of resistance to RIF. A majority (50-95%) of INH resistant strands carry a mutation in codon 315 of the katG gene and 20-35% carry mutations in the inhA promoter region. Less common mutations responsible for the remaining percentages are also mostly well characterized. This system provides an ideal test case for nucleic acid circuitry because diagnosis of resistance to either antibiotic requires analysis of multiple point mutations in parallel.

A logic circuit diagram for an example diagnostic circuit is shown in FIG. 14B. An amplifier module, as described in Example 1, may be used for detection of each specific mutation. Multiple different mutations that can independently lead to resistance to the same antibiotic are connected with each other in a logical OR clause. Amplifier modules are also used to detect markers associated with the M. tuberculosis complex. Detection of multiple markers can reduce false positive detection rates if the markers are connected in a logical AND. To further simplify the readout, the output signal from the OR operation that tests for resistance may be combined with the result of the TB detection module. Implementation of logical AND and OR gates, using recently demonstrated seesaw gates and thresholds, were used for an experimental implementation of this circuit (Qian et al., 2011).

An important feature of the systems described above is the modularity and scalability of circuits. Additional analytes may be added to an existing circuit and the logic may be easily changed to reflect a different diagnostic need. For example, in the circuit of FIG. 14B it may be desirable to have an independent readout for the TB markers and not use the second layer of logic. In the longer term, the circuit may be extended to include additional markers associated with extensively drug resistant TB. For biosensing applications, DNA circuits should ideally be able to operate in blood serum and similarly complex environments and thus require sample pre-preparation. Very recently, a first example of a DNA strand displacement reaction in serum was demonstrated (Graugnard et al., 2011). Furthermore, there are several examples of DNA circuitry operating in total RNA (Seelig et al., 2006), cell extract (Zhang et al., 2007) and similar complex biochemical environments. The diagnostic circuit described above may be developed using relatively simple reaction environments and may be later tested directly in serum.

### Example 4

### Development of DNA Logic Circuits for Paper-Based Reactions and Visual Readout

In Examples 1 and 2, DNA circuit reactions were carried out in tube-based reactions by manual addition of reagents and were analyzed using a fluorescence detection instrument. In this Example, DNA logic circuits may be adapted to be compatible with paper devices appropriate for point-of-use applications. For detection, DNA circuit outputs may be labeled with gold nanoparticles for visual detection and tested using outputs from tube-based reactions; hybridization kinetics may be measured to determine times for nearly complete capture of the output DNA. For the reaction component, DNA logic circuits as fluid-phase reactions in a paper matrix may be tested. Logic components may also be immobilized onto paper for solid-phase logic reactions. Reaction kinetics may be measured by comparison to tube-based reactions. The DNA logic circuits may be designed with visible detection starting with simple logic circuits and progressing to more sophisticated circuits. For example, the TB assay from Example 2 may be used.

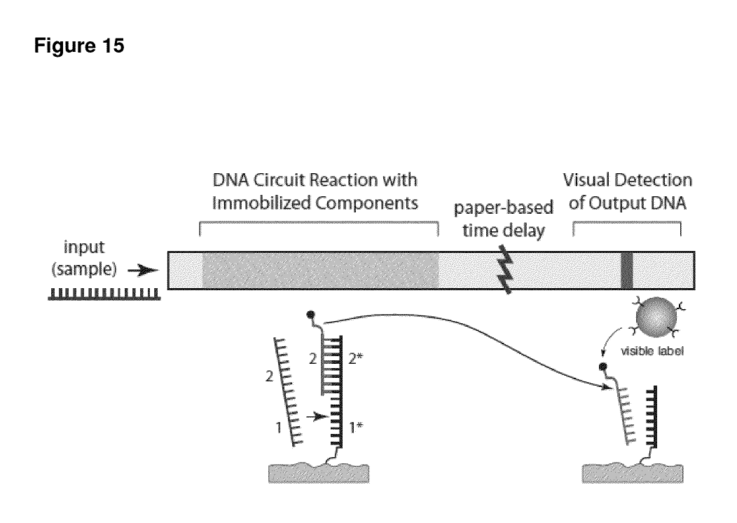

FIG. 15 shows a schematic of a device that motivates the fundamental work in this Example. The basic parts of the strip are a reaction zone and a detection zone for capture and visual detection of labeled DNA circuit outputs. In the reaction zone, the DNA circuit chemistry may be reacted in the fluid-phase or by immobilizing DNA logic components with pre-bound output DNA (this version is shown for the simplest one-input-one-output circuit, FIG. 15). The released output DNA can be captured at the detection line by hybridization to immobilized complementary probes. The reaction and detection zones can be separated by a paper-based time delay (see dissolvable delays discussed below) that can be programmed to match the reaction kinetics for automation of the two-step reaction. The two stages (detection and reaction) may be tested separately at first, and then demonstrated as an integrated system.

First, DNA may be captured and detected using visible labels. In the tube-based reactions of Examples 1 and 2, fluorophores or quenchers were conjugated to the output DNA; here, output DNA may include tags (e.g., FITC, Dig) that can be bound by gold nanoparticles (e.g., anti-FITC antibody, anti-Dig antibody). Capture probes may be purchased with poly-T tails which preferentially adsorb onto native nitrocellulose to provide preferential oriented immobilization (simply requires spotting and drying prior to use). The output DNA may be labeled in different ways: 1) mixing the DNA logic components with the label during the reaction, or 2) carrying out the reaction followed by mixing with the label. Detection may be tested for in both cases using reactions carried out in tubes.

The kinetics of hybridization capture may be tested by running strips and measuring the intensity of gold at the capture line (a flatbed scanner is routinely used to quantify signals). Exposure times for the hybridization reaction may be varied by simply changing the length of the detection strip (flow rate is inversely proportional to the strip length). A kinetic binding curve for the hybridization may be constructed. A time that allows nearly complete capture of the output DNA may be used for subsequent experiments. The high density of surface binding sites can lead to rapid capture, and surface capture sites are normally in large excess compared to analyte, so near 100% capture is typically achievable if given sufficient time. Capture times of less than 5 minutes should be achievable based on immunoassays and the similar binding kinetics for common hybridization reactions. The kinetics of this assay may be further optimized.

DNA Logic Reactions in Paper.

The DNA logic reaction may be carried out as a fluid-phase reaction (similar to the tube-based reaction but with fluid held in paper) or as a solid-phase reaction with immobilized components (an elegant but untested approach). The reaction kinetics for these two approaches may be measured by comparison to the tube-based reactions. Of note, it is expected that immobilization could have significant effects on the reaction kinetics due to constraints on reactant orientation. Real-time measurements of the fluorescence signal may be made directly on the nitrocellulose by using long-wavelength probes to avoid the modest auto fluorescence of nitrocellulose. Alternatively, an end-point measurement at each time point may be made using the visual readout developed above; after a given reaction time, the reaction strip may be manually connected to a detection strip (this approach has been used for development of immunoassays and nucleic acid assays).

Reaction kinetics may be evaluated using the same general framework used in Examples 1 and 2. For the case of immobilized logic circuits, the effective volumetric concentration may be estimated from the known binding capacity for nitrocellulose. Quantitative evaluation of the simplest strand displacement reaction for a single input and single output (FIG. 15) may be performed first, and then may progress to more sophisticated DNA circuit reactions.

The ability to immobilize functional DNA circuits allows for the manipulation of reaction processes that is not possible in homogeneous tube-based reactions; reactions can be spatially-separated (with potential for multiple stage reactions) and exposure of the sample to reaction components can be controlled by flow. DNA circuit components may be biotinylated to allow for immobilization on nitrocellulose with adsorbed streptavidin. It is expected that immobilization and labeling will decrease the rates of strand displacement reactions due to constraints in reactant orientation. A reaction curve may be generated for direct comparison to benchtop reactions via R and k (where R is the reaction rate and k is the reaction rate coefficient with units of concentration/time). Reaction kinetics may be evaluated for different lengths of the poly-T tail for immobilization. A comparison of both R and k should allow for discernment of the fundamental effects due to immobilization (effect on k) versus combined effects of immobilization and high reaction site density (effect on R).

Demonstration of a Paper Strip for Reaction and Visual Detection.

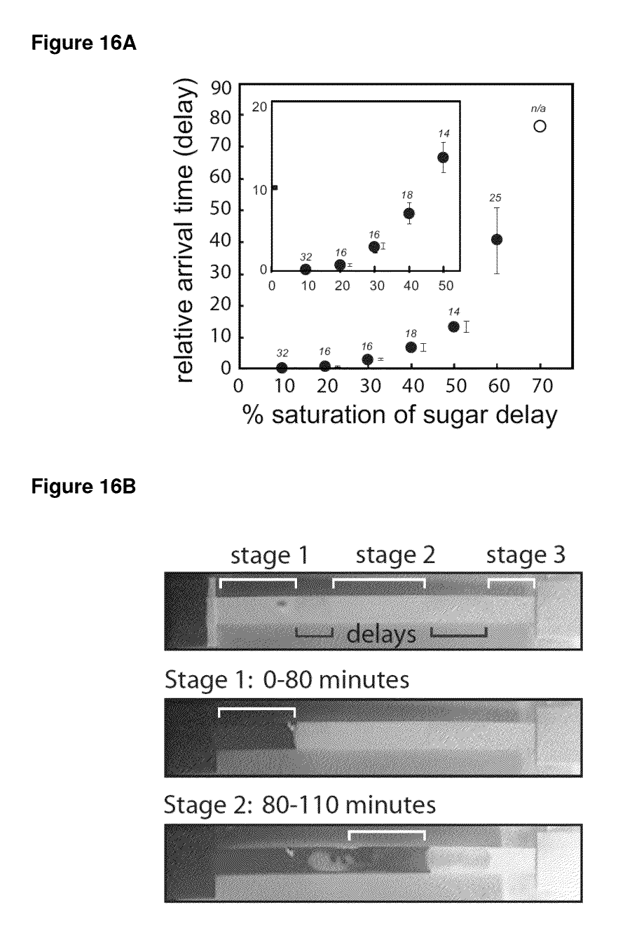

The reagents developed above may be used to demonstrate a simple strip visual readout of DNA circuit reactions like that shown in FIG. 15. The timing for each stage may be set based on the reaction and capture kinetics measured above. Paper-based delay timers using dried sugar solutions were already developed. FIG. 16A shows the delay time (relative to an untreated paper strip); delays can be selected from minutes to over an hour by using solutions of different saturation levels. FIG. 16B demonstrates the use of delay timers to stage fluids in different sections of a simple paper strip, much like the vision image in FIG. 15. Liquid reagents may be used to demonstrate an integrated system. The ability to dry down and rehydrate functional DNA circuit reagents may also be tested; sugar solutions are routinely used as a matrix to store functional reagents in dry form. The simplest DNA logic circuit may be tested in addition to the TB diagnostic analysis from Example 2 using this paper strip based methodology.

In conclusion, DNA-based molecular amplifiers may be designed with single mismatch specificity, to integrate multiple amplifiers into multi-input circuits for diagnostic applications, and to demonstrate circuit operation in a paper-based lateral flow device. This work lays the groundwork for building inexpensive and easy-to-use point-of-care diagnostic devices.

### REFERENCES

The references, patents and published patent applications listed below, and all references cited in the specification above are hereby incorporated by reference in their entirety, as if fully set forth herein.
Abebe, G., F. Paasch, L. Apers, L. Rigouts, and R. Colebunders, Tuberculosis drug resistance testing by molecular methods: Opportunities and challenges in resource limited settings, J. Microbiol. Methods. 84, 155 (2010).
Aldaye, F. A., Palmer, A. L. & Sleiman, H. F. Assembling materials with DNA as the guide. Science 321, 1795-1799 (2008).
Alvarez-Garcia, I. and Miska, E. A., MicroRNA functions in animal development and human disease, Development 132, 4653 (2005).
Ashkenasy, G. & Ghadiri, M. R. Boolean Logic Functions of a Synthetic Peptide Network J. Am. Chem. Soc., 126, 11140-11141 (2004).
Bang, D., A. B. Andersen, and V. O. Thomsen, Rapid genotypic detection of rifampin- and isoniazidresistantn Mycobacterium tuberculosis directly in clinical specimens, J. Clin. Microbiol. 44, 2605 (2006).
Bang, H., Park, S., Hwang, J. & Lee1, H. Improved rapid molecular diagnosis of multidrug-resistant tuberculosis using a new reverse hybridization assay, REBA MTB-MDR. Journal of Medical Microbiology 60, 14471454 (2011).
Bartel, D. P. MicroRNAs: target recognition and regulatory functions. Cell 136, 215-233 (2009).
Benenson, Y., Gil, B., Ben-Dor, U., Adar, R. & Shapiro, E. An autonomous molecular computer for logical control of gene expression. Nature 429, 423-429 (2004).
Benenson, Y., Paz-Elizur, T., Adar, R., Keinan, E., Livneh, Z. & Shapiro, E. Programmable and autonomous computing machine made of biomolecules. Nature 414, 430-434 (2001).
Bois, J. S., Venkataraman, S., Choi, H. M. T., Spakowitz, A. J., Wang, Z. G. & Pierce, N. A. Topological Constraints in Nucleic Acid Hybridization Kinetics. Nucleic Acids Res. 33, 4090-4095 (2005).
Bonnet, G., Tyagi, S., Libchaber, A. & Kramer, F. R. Thermodynamic basis of the enhanced speci_city of structured DNA probes. Proc. Nat. Acad. Sci. 96, 6171-6176 (1999).
Brossier, F., N. Veziris, C. Truffot-Pernot, V. Jarlier, and W. Sougakoff, Performance of the genotype MTBDR line probe assay for detection of resistance to rifampin and isoniazid in strains of Mycobacterium tuberculosis with low- and high-level resistance, J. Clin. Microbiol. 44 3659 (2006).
Calin, G. A. and Croce, C. M., MicroRNA-cancer connection: the beginning of a new tale, Cancer research 66, 7390 (2006).
Calin, G. A. Human microRNA genes are frequently located at fragile sites and genomic regions involved in cancers Proc. Nat. Acad. Sci. 101, 29993004 (2004).
Cardelli, L. Strand algebras for DNA computing. Lect. Notes. Comput. Sci. 5877, 12-24 (2008).
Carter, D. J. and R. B. Cary, Lateral flow microarrays: a novel platform for rapid nucleic acid detection based on miniaturized lateral flow chromatography, Nucleic Acids Res. (2007).
Cavusoglu, C., A. Turhan, P. Akinci, and I. Soyler, Evaluation of the genotype MTBDR assay for rapid detection of rifampin and isoniazid resistance in Mycobacterium tuberculosis isolates, J. Clin. Microbiol. 44, 2338 (2006).
Chen, X., Ba, Y., Ma, L., Cai, X., et al. Characterization of microRNAs in serum: a novel class of biomarkers for diagnosis of cancer and other diseases, Cell Research 18, 997 (2008).
Corstjens, P., M. Zuiderwijk, A. Brink, S. Li, H. Feindt, R. S. Neidbala, and H. Tanke, Use of upconverting phosphor reporters in lateral-flow assays to detect specific nucleic acid sequences: A rapid, sensitive DNA test to identify human papillomavirus type 16 infection, Clin. Chem. 47, 1885 (2001).
Corstjens, P., M. Zuiderwijk, M. Nilsson, H. Feindt, R. S. Niedbala, and H. J. Tanke, Lateral-flow and up-converting phosphor reporters to detect single-stranded nucleic acids in a sandwich-hybridization assay, Anal. Biochem. 312, 191 (2003).
de Silva, A. P. & Uchiyama, S. Molecular logic and computing Nat. Nanotechnol. 2, 399-410 (2007).
Dirks, R. M., Lin, M., Winfree, E., Pierce, N. A. Paradigms for computational nucleic acid design. Nucl. Acids Res. 32, 1392-1403 (2004).
Dirks, R. M., Bois, J. S., Schae_er, J. M., Winfree, E. & Pierce, N. A. Thermodynamic Analysis of Interacting Nucleic Acid Strands. SIAM Review 49, 65-88 (2007).
Dungchai, W., O. Chailapakul, and C. S. Henry, Use of multiple colorimetric indicators for paper-based microfluidic devices, Analytica Chimica Acta 674 227 (2010).
Edwards, K. A. and A. J. Baeumner, Optimization of DNA-tagged dye-encapsulating liposomes for lateral-flow assays based on sandwich hybridization, Anal. Bioanal. Chem. 386, 1335 (2006).
Epstein, I. R. & Pojman, J. A. An Introduction to Nonlinear Chemical Dynamics. Oxford University Press, New York, N. Y. (1998).
Esquela-Kerscher, A. and Slack, F. J., Oncomirs: microRNAs with a role in cancer, Nature Reviews Cancer 6, 259 (2006).
Fenton, E., M. Mascarenas, G. Lopez, and S. Sibbett, Multiplex lateral-flow test strips fabricated by two-dimensional shaping, Applied Materials and Interfaces 1 124 (2009).
Frezza, B. M., Cockroft, S. L. & Ghadiri, M. R. Modular multi-level circuits from immobilized DNA-based logic gates. J. Am. Chem. Soc. 129, 14875-14879 (2007).
Friedrich, S. O., A. Venter, X. A. Kayigire, R. Dawson, P. R. Donald, and A. H. Diacon, Suitability of Xpert MTB/RIF and Genotype MTBDRplus for Patient Selection for a Tuberculosis Clinical Trial, J. Clin. Microbiol. 49, 2827 (2011).
Fu, E., B. Lutz, P. Kauffman, and P. Yager, Controlled reagent transport in disposable 2D paper networks, Lab on a Chip 10, 918 (2010a).
Fu, E., P. Kauffman, B. Lutz, and P. Yager, Chemical signal amplification in two-dimensional paper networks, Sensors and Actuators B-Chemical 149 325 (2010b).
Fu, E., S. A. Ramsey, P. Kauffman, B. Lutz, and P. Yager, Transport in two-dimensional paper networks, Microfluidics and Nanofluidics DOI 10.1007/s10404-010-0643-y (2010c).
Graugnard, E., Cox, A., Lee, J., Jorcyk, C., Yurke, B., and Hughes, W. Operation of a DNA-based autocatalytic network in serum, Lecture Notes in Computer Science 6518, 83 (2011).
Green, S. J., Lubrich, D. & Turberfield, A. J. DNA hairpins: fuel for autonomous DNA devices. Biophysical Journal 91, 2966-2975 (2006).
Gunderson, K. L., Steemers, F. J., Lee, G., Mendoza, L. G. & Chee, M. S. A genome-wide scalable SNP genotyping assay using microarray technology. Nature Biotechnology 37, 549-554 (2005).
Hillemann, D., M. Weizenegger, T. Kubica, E. Richter, and S, Niemann, Use of the genotype MTBDR assay for rapid detection of rifampin and isoniazid resistance in Mycobacterium tuberculosis complex isolates, J. Clin. Microbiol. 43, 3699 (2005).
Hillemann, D., S. Rusch-Gerdes, and E. Richter, Evaluation of the GenoType MTBDRplus assay for rifampin and isoniazid susceptibility testing of Mycobacterium tuberculosis strains and clinical specimens, J. Clin. Microbiol. 45, 2635 (2007). infectious diseases, Trends in biotechnology 29, 240 (2011).
Ioannou, Carbon nano-strings as reporters in lateral flow devices for DNA sensing by hybridization, Anal. Bioanal. Chem. 400, 1145 (2011).
Isaacs, F. J., Dwyer, D. J. & Collins, J. J. RNA synthetic biology. Nat. Biotechnol. 24, 545-554 (2006).
Kalogianni, D. P., L. M. Boutsika, P. G. Kouremenou, T. K. Christopoulos, and P. C. Kauffman, P., E. Fu, B. Lutz, and P. Yager, Visualization and measurement of flow in two-dimensional paper networks, Lab on a Chip 10, 2614 (2010).
Kim, J., White, K. S. & Winfree, E. Construction of an in vitro bistable circuit from synthetic transcriptional switches. Molecular Systems Biology 2, 68-79 (2006).
Kim, S. & Misra A. SNP genotyping: technologies and biomedical applications. Annu. Rev. Biomed. Eng. 9, 289-320 (2007).
Koltai, H. & Weingarten-Baror, C. Speci_city of DNA microbarray hybridization: characterization, e_ectors, and approaches for data correction. Nucleic Acids Research 36, 2395-2405 (2008).
Krishnan, Y. and Simmel, F. C., Nucleic Acid Based Molecular Devices 50, 3124 (2011). Lawrie, C. H., Gal, S., Dunlop, H. M., Pushkaran, B., et al., Detection of elevated levels of tumour associated microRNAs in serum of patients with diffuse large B-cell lymphoma, British journal of haematology 141 672 (2008).
Lederman, H., Macdonald, J., Stephanovic, D. & Stojanovic, M. N. Deoxyribozyme-based three-input logic gates and construction of a molecular full adder. Biochemistry 45, 1194-1199 (2006).
Li, B., Ellington, A. D. & Chen, X. Rational, modular adaptation of enzyme-free DNA circuits to multiple detection methods. Nucleic Acids Research 39, e110 (2011).
Li, Q., Luan G., Guo, Q. & Liang J. A new class of homogeneous nucleic acid probe based on specific displacement hybridization, Nucleic Acids Res. 30, e5 (2002).
Li, X., J. F. Tian, and W. Shen, Quantitative biomarker assay with microfluidic paper-based analytical devices, Analytical and Bioanalytical Chemistry 396, 495 (2010).
Liu, J. and Cao, Z. and Lu, Y., Functional Nucleic Acid Sensors, Chem. Rev 109, 1948 (2009).
Lu J. et al. MicroRNA expression profiles classify human cancers. Nature 435, 834-838 (2005).
Lu, Y. & Liu, J. Functional DNA nanotechnology: emerging applications of DNAzymes and aptamers. Curr. Opin. Biotechnol. 17, 580-588 (2006).
Makinen, J., H. J. Marttila, M. Marjamaki, M. K. Viljanen, and H. Soini, Comparison of two commercially available DNA line probe assays for detection of multidrug resistant Mycobacterium tuberculosis J. Clin. Microbiol. 44, 350 (2006).
Mao, X., Y. Q. Ma, A. G. Zhang, L. R. Zhang, L. W. Zeng, and G. D. Liu, Disposable Nucleic Acid Biosensors Based on Gold Nanoparticle Probes and Lateral Flow Strip, Anal. Chem. 81 1660 (2009).
Marras S. A., F. R. Kramer & S. Tyagi. E_ciencies of fluorescence resonance energy transfer and contact-mediated quenching in oligonucleotide probes. Nucleic Acids Research 30, e122 (2002).
Martinez, A. W., S. T. Phillips, E. Carrilho, S. W. Thomas, H. Sindi, and G. M. Whitesides, Simple telemedicine for developing regions: Camera phones and paper-based microfluidic devices for real-time, off-site diagnosis, Analytical Chemistry 80, 3699 (2008).
Martinez, A. W., S. T. Phillips, M. J. Butte, and G. M. Whitesides, Patterned paper as a platform for inexpensive, low-volume, portable bioassays, Angewandte Chemie-International Edition 46 1318 (2007).
McNerney, R. and Daley, P., Towards a point-of-care test for active tuberculosis: obstacles and opportunities, Nature Reviews Microbiology 9, 204 (2011).
Miotto, P., F. Piana, V. Penati, F. Canducci, G. B. Migliori, and D. M. Cirillo, Use of genotype MTBDR assay for molecular detection of rifampin and isoniazid resistance in Mycobacterium tuberculosis clinical strains isolated in Italy, J. Clin. Microbiol. 44 2485 (2006).
Mitchell, P. S., Parkin, R. K., Kroh, E. M., Fritz, B. R. et al., Circulating microRNAs as stable blood-based markers for cancer detection, Proceedings of the National Academy of Sciences 105, 10513 (2008).
Navani, N. K. and Li, Y., Nucleic acid aptamers and enzymes as sensors, Current opinion in chemical biology 10, 272 (2006).
Ngom, B., Y. C. Guo, X. L. Wang, and D. R. Bi, Development and application of lateral flow test strip technology for detection of infectious agents and chemical contaminants: a review Anal. Bioanal. Chem. 397 1113 (2010).
Niemz, A., Ferguson, T. M., and Boyle, D. S., Point-of-care nucleic acid testing for Noguera, P., G. A. Posthuma-Trumpie, M. van Tuil, F. J. van der Wal, A. de Boer, A. Moers, and A. Van Amerongen, Carbon nanoparticles in lateral flow methods to detect genes encoding virulence factors of Shiga toxin-producing Escherichia coli, Anal. Bioanal. Chem. 399, 831 (2011).
O'Farrell, B., R. Wong, and H. Tse, Evolution in Lateral Flow-Based Immunoassay Systems Humana Press. p. 1-33. (2009).
Osborn, J., B. Lutz, E. Fu, P. Kauffman, D. Stevens, and P. Yager, Microfluidics without pumps: reinventing the T-sensor and H-filter in paper networks, Lab on a Chip DOI: 10.1039/c004821f (2010).
Panyutin, I. G. & Hsieh P. The kinetics of spontaneous DNA branch migration. Proc. Nat. Acad. Sci. 91, 2021-2025 (1994).
Parsons, L. M. P. L. M., A. Somoskovi, C. Gutierrez, E. Lee, C. N. Paramasivan, A. Abimiku, S. Spector, G. Roscigno, and J. Nkengasong, Laboratory Diagnosis of Tuberculosis in Resource-Poor Countries: Challenges and Opportunities, Clin. Microbiol. Rev. 24 314 (2011).
Peeling, R. W. and D. Mabey, Point-of-care tests for diagnosing infections in the developing world. Clinical Microbiology and Infection 16, 1062 (2010).
Penchovsky R. & Breaker R. R. Computational design and experimental validation of oligonucleotide sensing allosteric ribozymes. Nature Biotech. 23, 1424-1433 (2005).
Peyret, N. Prediction of Nucleic Acid Hybridization: Parameters and Algorithms. Doctoral thesis, Wayne State University. (2000)
Qian, L. and Winfree, E., Scaling Up Digital Circuit Computation with DNA Strand Displacement Cascades, Science 332, 1196 (2011).
Rothemund, P. W. K. Folding DNA to create nanoscale shapes and patterns. Nature 440, 297-302 (2005).
Roush, S, and Slack, F. J., The let-7 family of microRNAs, Trends in cell biology 18, 505 (2008).
Rule, G. S., R. A. Montagna, and R. A. Durst, Rapid method for visual identification of specific DNA sequences based on DNA-tagged liposomes, Clin. Chem., 42 1206 (1996).
Saiki, R. K., Gelfand, D. H., Sto_el, S., Scharf, S. J., Higuchi, R., Horn, G. T., Mullis, K. B. & Erlich, H. A. Primer-directed enzymatic ampli_cation of DNA with a thermostable DNA polymerase. Science 239, 487-491 (1988).
SantaLucia, J. & Hicks, D. The Thermodynamics of DNA Structural Motifs. Ann. Rev. Biochem. 33, 415-440 (2004).
Schena, M., Shalon, D., Davis, R. W. & Brown, P. O. Quantitative Monitoring of Gene Expression Patterns with a Complementary DNA Microarray. Science 270, 467-470 (1995).
Seelig, G., Soloveichik, D., Zhang, D. Y. & Winfree, E. Enzyme-free nucleic acid logic circuits. Science 314, 1585-1588 (2006a).
Seelig, G., Yurke, B. & Winfree, E. Catalyzed Relaxation of a Metastable DNA fuel. J. Am. Chem. Soc. 128, 12211-12220 (2006b).
Seeman, N. C., Nanomaterials Based on DNA. Annu. Rev. Biochem. 79, 65-87 (2010).
Shih, W. M. & Lin, C. Knitting complex weaves with DNA origami. Curr. Opin. Struct. Biol. 20, 276-282 (2010).
Sidransky, D., Emerging molecular markers of cancer, Nature Reviews Cancer 2, 210 (2002).
Simpson, Z. B., Tsai, T. L., Nguyen, N., Chen, X. & Ellington, A. D. Modelling amorphous computations with transcription networks. J. R. Soc. Interface 6, S523-S533 (2009).
Soloveichik, D., Seelig, G. & Winfree, E. DNA as a universal substrate for chemical kinetics. Proc. Nat. Acad. Sci. 107, 5393-5398 (2010).
Stojanovic, M. N. & Stefanovic D. A deoxyribozyme-based molecular automaton. Nat. Biotechnol. 21, 1069-1074 (2003).
Stojanovic, M. N., Mitchell, T. E. & Stefanovic D. Deoxyribozyme-based logic gates. J. Am. Chem. Soc. 124, 3555-3561 (2002).
Subramanian, H. K. K., Chakraborty, B., Sha, R. & Seeman, N. C. The label-free unambiguous detection and symbolic display of single nucleotide polymorphisms on DNA origami. Nano Letters 11, 910-913 (2010).
Takahashi, K., Yaegashi, S., Kameda, A. & Hagiya, M. Chain Reaction Systems Based on Loop Dissociation of DNA. Led. Notes. Comput. Sci. 3892, 347-358 (2006).
Tan, Z. J. & Chen, S. J. Nucleic Acid Helix Stability: Effects of Salt Concentration, Cation Valence and Size, and Chain Length. Biophys. J. 90, 1175-1190 (2006).
Thompson, B. J., Camien, M. N. & Warner, R. C. Kinetics of branch migration in double-stranded DNA. Proc. Nat. Acad. Sci. 73, 2299-2303 (1976).
Tsang, J. C. H. and Dennis Lo, Y. M., Circulating nucleic acids in plasma/serum, Pathology 39, 197 (2007).
Tsourkas, A., Behlke, M. A., Rose, S. D. & Bao, G. Hybridization kinetics and thermodynamics of molecular beacons. Nucleic Acids Research 31, 1319-1330 (2003).
Turberfield, A. J. et al. DNA Fuel for Free-running Nanomachines. Phys. Rev. Lett. 90, 118102 (2003).
Tyagi, S. & Kramer, F. R. Molecular beacons: probes that fluoresce upon hybridization. Nature Biotechnology 14, 303-308 (1996).
Urdea, M., L. A. Penny, S. S. Olmsted, M. Y. Giovanni, P. Kaspar, A. Shepherd, P. Wang, W., W. Y. Wu, W. Wang, and J. J. Zhu, Tree-shaped paper strip for semiquantitative colorimetric detection of protein with self-calibration, Journal of Chromatography A 1217 3896 (2010).
Weinberg, R. A., The biology of cancer, Garland Science New York (2002).
Willner, I., Shlyahovsky, B., Zayats, M. & Willner, B. DNAzymes for sensing, nanobiotechnology, and logic gate applications. Chem. Soc. Rev. 37, 1153-1165 (2008).
Wilson, C. A. Dahl, S. Buchsbaum, G. Moeller, and D. C. Hay Burgess, Requirements for high impact diagnostics in the developing world, Nature 444 Suppl 1, 73 (2006).
Win, M. N. & Smolke, C. D. Higher-order cellular information processing with synthetic RNA devices. Science 322, 456-460 (2008).
Xiao, Y., Plakos, K. J. I., Lou, X., White, R. J., Qian, J., Plaxco, K. W. & Soh, H. T. Fluorescence detection of single-nucleotide polymorphisms with a single, self-complementary, triple-stem DNA probe, Angew. Chemie Int. Ed. 48, 4354-4358 (2009).
Yager, P., G. J. Domingo, and J. Gerdes, Point-of-care diagnostics for global health, Annu Rev Biomed Eng 10 107 (2008).
Yager, P., T. Edwards, E. Fu, K. Helton, K. Nelson, M. Tam, and B. Weigl, Microfluidic diagnostic technologies for global public health, Nature Insight 442 412 (2006)
Yashin, R., Rudchenko, S. & Stojanovic, M. N. Networking particles over distance using oligonucleotide based devices. J. Am. Chem. Soc. 129, 15581-15584 (2007).
Yeh, B. J. & Lim, W. A. Synthetic biology: lessons from the history of synthetic organic chemistry. Nat. Chem. Biol. 3, 521-525 (2007).
Yin, P., Choi, H. M. T., Calvert, C. R. & Pierce, N. A. Programming biomolecular self-assembly pathways. Nature 451, 318-322 (2008).
Yurke, B. & Mills, A. P. Using DNA to Power Nanostructures, Genetic Programming and Evolvable Machines 4, 111-122 (2003).
Zhang, D. Y. & Seelig, G., Dynamic DNA Nanotechnology Using Strand Displacement Reactions. Nat. Chem. 3, 103 (2011a).
Zhang, D. Y. and Seelig, G., DNA-based Fixed Gain Amplifiers and Linear Classifier Circuits, DNA 16 Lecture Notes in Computer Science 6518, 176 (2011b).
Zhang, D. Y. & Winfree, E. Control of DNA Strand Displacement Kinetics Using Toehold Exchange. J. Am. Chem. Soc. 131, 17303-17314 (2009).
Zhang, D. Y. & Winfree, E. Robustness and modularity properties of a non-covalent DNA catalytic reaction. Nucleic Acids Res. 38, 4182-4197 (2010).
Zhang, D. Y., Turberfield, A. J., Yurke, B. & Winfree, E. Engineering entropy-driven reactions and networks catalyzed by DNA. Science 318, 1121-1125 (2007).
Zhang, D. Z, Chen. S. X. &Yin, P. Optimizing the specificity of nucleic acid hybridization Nature Chemistry 4, 208-214 (2012).
Zhao, W. A., M. M. Ali, S. D. Aguirre, M. A. Brook, and Y. F. Li, Paper-Based Bioassays Using Gold Nanoparticle Colorimetric Probes, Analytical Chemistry 80 8431 (2008).

What is claimed is:

1. A strand displacement system comprising:
a first nucleic acid catalyst molecule;
a nucleic acid gate molecule, wherein the first nucleic acid catalyst molecule binds the nucleic acid gate molecule forming a nucleic acid gate-catalyst complex and releases an output molecule; and
a nucleic acid sink molecule, which sequesters a putative second nucleic acid catalyst, wherein the second nucleic acid catalyst differs from the first nucleic acid catalyst molecule by at least one nucleotide.

2. The strand displacement system of claim 1, wherein the first nucleic acid catalyst comprises a biomarker of interest.

3. The strand displacement system of claim 2, wherein the biomarker of interest is a DNA or RNA molecule.

4. The strand displacement system of claim 1, wherein the first nucleic acid catalyst is a nucleic acid aptamer which binds an amino acid-based biomarker of interest.

5. The strand displacement system of claim 1, further comprising a nucleic acid fuel molecule, wherein the nucleic acid fuel molecule binds the nucleic acid gate/catalyst complex and releases the first nucleic acid catalyst molecule.

6. The strand displacement system of claim 1, wherein the output molecule is conjugated to a detection moiety to produce a detectable signal.

7. The strand displacement system of claim 6, wherein the detection moiety is a fluorophore.

8. The strand displacement system of claim 1, further comprising a paper-based diagnostic tool, which comprises
a set of reaction components attached to at least one reaction zone, wherein the set of reaction components includes the nucleic acid gate molecule and the nucleic acid sink molecule; and
a set of output capture probes attached to at least one detection zone, wherein the output capture probes are complementary to the output molecule released by the nucleic acid gate molecule.

9. The strand-displacement system of claim 8, wherein the nucleic acid gate molecule binds the first nucleic acid catalyst molecule, resulting in release of the output molecule.

10. The strand-displacement system of claim 9, wherein the output molecule binds the output capture probe to produce a detectable signal in a detection zone.

11. The strand-displacement system of claim 10, wherein the detectable signal is produced by a fluorophore or a gold nanoparticle.

12. The strand-displacement system of claim 8, wherein the paper device comprises nitrocellulose.

13. The strand-displacement system of claim 8, wherein the set of reaction components further comprises a fuel molecule.

14. The strand-displacement system of claim 8, wherein the paper device further comprises a time delay zone.

15. A method for detecting a biomarker of interest in a biological sample comprising:
exposing the biological sample which contains or is suspected of containing a first nucleic acid catalyst molecule to a reaction zone of a paper-based diagnostic tool, wherein the first nucleic acid catalyst molecule comprises the biomarker of interest and the reaction zone comprises a set of reaction components which includes a nucleic acid gate molecule and a nucleic acid sink molecule, and;
detecting the biomarker of interest by visualizing a change in signal in a detection zone of the paper-based diagnostic tool, wherein the change in signal is produced when an output molecule binds a nucleic acid capture molecule that is attached to the detection zone.

16. The method of claim 15, wherein the nucleic acid gate molecule comprises the output molecule conjugated to a fluorophore and a toehold strand conjugated to a quencher.

17. The method of claim 16, wherein the toehold strand of the nucleic acid gate molecule binds the first nucleic acid catalyst molecule, resulting in release of the output molecule.

18. The method of claim 15, wherein the a nucleic acid capture molecule comprises a gold nanoparticle.

19. The paper-based diagnostic tool of claim 10, wherein the nucleic acid sink molecule sequesters a putative second nucleic acid catalyst, and wherein the second nucleic acid catalyst differs from the first nucleic acid catalyst molecule by at least one nucleotide.

20. The paper-based diagnostic tool of claim 8, wherein the set of reaction components further comprises a fuel molecule.


SEQUENCE LISTING (24 sequences)

SEQ ID NO: 1 - RNA, 22 nt, Artificial Sequence
  Synthetic oligonucleotide let-7a
ugagguaguagguuguauaguu

SEQ ID NO: 2 - RNA, 22 nt, Artificial Sequence
  Synthetic oligonucleotide let-7b
ugagguaguagguugugugguu

SEQ ID NO: 3 - RNA, 22 nt, Artificial Sequence
  Synthetic oligonucleotide let-7c
ugagguaguagguuguaugguu

SEQ ID NO: 4 - RNA, 22 nt, Artificial Sequence
  Synthetic oligonucleotide let-7d
agagguaguagguugcauaguu

SEQ ID NO: 5 - RNA, 22 nt, Artificial Sequence
  Synthetic oligonucleotide let-7e
ugagguaggagguuguauaguu

SEQ ID NO: 6 - RNA, 22 nt, Artificial Sequence
  Synthetic oligonucleotide let-7f
ugagguaguagauuguauaguu

SEQ ID NO: 7 - RNA, 22 nt, Artificial Sequence
  Synthetic oligonucleotide let-7g
ugagguaguaguuuguacaguu

SEQ ID NO: 8 - RNA, 22 nt, Artificial Sequence
  Synthetic oligonucleotide let-7i
ugagguaguaguuugugcuguu

SEQ ID NO: 9 - DNA, 88 bp, M. tuberculosis
ctgagccaattcatggaccagaacacccgctgtcggggttgacccacaagcgccgactgtcggcgctggggcccggcggtctgtcacg

SEQ ID NO: 10 - DNA, 13 bp, Artificial Sequence
  Synthetic oligonucleotide
gtggtgtgtgtat

SEQ ID NO: 11 - DNA, 13 bp, Artificial Sequence
  Synthetic oligonucleotide
gtggtctgtgtat

SEQ ID NO: 12 - DNA, 13 bp, Artificial Sequence
  Synthetic oligonucleotide
atacacacaccac

SEQ ID NO: 13 - DNA, 14 bp, Artificial Sequence
  Synthetic oligonucleotide
ggtggtgtgtgtat

SEQ ID NO: 14 - DNA, 14 bp, Artificial Sequence
  Synthetic oligonucleotide
atacacacaccacc

SEQ ID NO: 15 - DNA, 14 bp, Artificial Sequence
  Synthetic oligonucleotide
ggtggtgtatgtgt

SEQ ID NO: 16 - DNA, 14 bp, Artificial Sequence
  Synthetic oligonucleotide
ggtggtgtatgtat

SEQ ID NO: 17 - DNA, 23 bp, Artificial Sequence
  Synthetic oligonucleotide
ccaaggtggtgtgtgtatagtga

SEQ ID NO: 18 - DNA, 23 bp, Artificial Sequence
  Synthetic oligonucleotide
agtgaatacacacaccaccttgg

SEQ ID NO: 19 - DNA, 23 bp, Artificial Sequence
  Synthetic oligonucleotide
tcactatacacacaccaccaacc

SEQ ID NO: 20 - DNA, 23 bp, Artificial Sequence
  Synthetic oligonucleotide
ggttggtggtgtgtgtattcact

SEQ ID NO: 21 - DNA, 23 bp, Artificial Sequence
  Synthetic oligonucleotide
ggttggtggtgtatgtgttcact

SEQ ID NO: 22 - DNA, 23 bp, Artificial Sequence
  Synthetic oligonucleotide
ggttggtggtgtatgtattcact

SEQ ID NO: 23 - DNA, 23 bp, Artificial Sequence
  Synthetic oligonucleotide
ccaaggtggtctgtgtatagtga

SEQ ID NO: 24 - DNA, 23 bp, Artificial Sequence
  Synthetic oligonucleotide
agtgaatacacagaccaccttgg